(19) United States
(12) Patent Application Publication
(10) Pub. No.: US 2006/0216294 A1
(43) Pub. Date: Sep. 28, 2006

(54) Method of modulation

(76) Inventors: Ian Stuart McLennan, Dunedin (NZ); Kyoko Koishi, Dunedin (NZ); Pei-Yu Wang, Wpy Dunedin (NZ)

Correspondence Address: HAYNES AND BOONE, LLP, 901 MAIN STREET, SUITE 3100, DALLAS, TX, 75202, US

(21) Appl. No.: 11/229,415

(22) Filed: Sep. 16, 2005

(30) Foreign Application Priority Data
Mar. 24, 2005 (NZ) 539036

(51) Int. Cl. A61K 39/395; A61K 38/54

(52) U.S. Cl. 424/145.1; 514/12

(57) ABSTRACT

Disclosed are methods for modulating neuron activity, and in particular, for modulating motor neuron function and activity, particularly for use in the treatment, prevention, and/or amelioration of symptoms of one or more motor neuron conditions or diseases in a mammal. Also disclosed are compositions comprising one or more agonists or antagonists of a mammalian Müllerian Inhibitory Substance receptor polypeptide, as well as pharmaceutical formulations, isolated host cells, and therapeutic and/or diagnostic kits that comprises such agonists or antagonists.

### BACKGROUND OF THE INVENTION

The present application claims priority to New Zealand Patent Application No. 539036, filed Mar. 24, 2005, the entire contents of which is specifically incorporated herein by reference.

### FIELD OF THE INVENTION

The present invention relates generally to the field of molecular biology, and to a method of modulating neuron activity and in particular to modulating motor neuron function and activity, particularly although by no means exclusively to treat, prevent, and/or ameliorate the symptoms of one or more motor neuron diseases in a mammal.

### DESCRIPTION OF RELATED ART

Motor neuron diseases encompass a range of conditions that involve a loss of brain and spinal cord motor neuron function. Motor neurons are the nerve cells that link the brain to muscles. Degeneration of the motor neurons leads to weakness and wasting of muscles. This generally occurs in the arms or legs but can also occur in the muscles of the face and throat, causing problems with speech and difficulty chewing and swallowing.

Common motor neuron diseases include amyotrophic lateral sclerosis (ALS or Lou Gehrig's Disease); progressive muscular atrophy; and postpolio syndrome. Motor neuron disease is generally a steadily progressive disease and can progress to death through paralysis.

The most economically important motor neuron disease is sarcopenia, an age-related loss of muscle function resulting in old-age frailty. In the United States, sarcopenia accounts for 1.5% of the total direct health costs and is the leading reason why people need to go into care.

There is currently no cure for motor neuron disease. Therapy with neuroprotective agents such as riluzole and gabapentin has been attempted in the hope of slowing neuronal loss, by activating either the repair and/or survival mechanisms. Nutritional supplements (e.g., creatine) have also been trialed in an attempt to preserve muscle bulk. Such trials have met with only limited success.

It is an object of the present invention to provide a method of modulating motor neuron activity which goes at least some way towards addressing the problems disclosed above, or to at least provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

Further aspects and advantages of the present invention will become apparent from the ensuing description which is given by way of example only.

### SUMMARY OF THE INVENTION

The present invention overcomes a variety of limitations and deficiencies inherent in the prior art by providing novel and useful compositions and methods for treating, preventing, and/or ameliorating the symptoms of one or more motor neuron deficits in an affected animal. As noted above, and as detailed hereinbelow, the present inventors have surprisingly found that an agonist or antagonist of Müllerian Inhibitory Substance receptor is effective in modulating motor neuron disease.

Müllerian Inhibitory Substance (MIS) is a member of the transforming growth factor-β (TGFβ) superfamily of growth factors. MIS, also known as anti-Müllerian hormone (AMH), is required for normal male reproductive tract development. In the absence of MIS, males become partially feminized. The role of MIS has previously been thought to be restricted to reproductive tissues. The present invention is thus directed to this surprising finding.

According to one aspect of the present invention there is provided a method of modulating motor neuron activity in an animal in need thereof, and particularly in a mammal in need thereof, said method comprising at least the step of administering or providing to said animal or mammal, patient an effective amount of at least one Müllerian inhibitory substance (MIS) receptor agonist or antagonist. In particular embodiments the mammal is a human that has, is suspected of having, or is at risk for developing altered motor neuron activity.

The present invention also provides a method of enhancing neuronal cell survival in a patient in need thereof, the method comprising administering an effective amount of at least one MIS receptor agonist or antagonist to said animal.

The invention also provides a method of treating, preventing, or ameliorating the symptoms of one or more conditions, diseases, disorders, or dysfunctions that is characterized in part by motor neuron cell death or impairment. In an overall sense, the methods of the invention may comprise, for example, administering to a patient in need thereof, a therapeutically-effective amount of at least one MIS receptor agonist or antagonist, for a time and under conditions sufficient to treat, prevent, or ameliorate the symptoms of one or more such conditions, diseases, or disorders.

In illustrative embodiments the motor neuron disease is amyotrophic lateral sclerosis (ALS), also known as Lou Gehrig's Disease. However, this should not be seen a limitation, as the present invention is applicable to other motor neuron diseases, including for example, but not limited to, progressive muscular atrophy, postpolio syndrome and sarcopenia, the latter of which results in old-age frailty.

The present invention also provides a method of modulating neuronal cell survival in a patient following injury to the brain, comprising administering to said patient an effective amount of an MIS receptor agonist or antagonist.

The present invention also provides a method of enhancing neuronal cell survival in vitro, the method comprising culturing the neuronal cells in the presence of at least one MIS receptor agonist or antagonist.

Exemplary MIS receptors include, but are not limited to, a type II receptor (MISRII), a type I receptor, or biological functional equivalents, or alternatively may comprise a combination of two or more such receptors. In particular illustrative embodiments, the MIS receptor is a mammalian receptor, and more particularly, a mammalian type II receptor (MISRII).

Preferably the MISRII agonist or antagonist is Müllerian inhibitory substance (MIS), a functional derivative or analog thereof, or an antibody or antigen binding fragment that specifically binds thereto.

The present invention also provides the use of at least one MIS receptor agonist or antagonist that is capable of interacting with an MIS receptor, in the manufacture of pharmaceutical formulations, including, for example, medicaments for modulating neuron activity.

In another embodiment the invention also provides for the use of at least one MIS receptor agonist or antagonist in the manufacture of a medicament to treat or prevent motor neuron disease in an animal, including for example, diseases such as ALS, progressive muscular atrophy, postpolio syndrome and/or sarcopenia.

The present invention also provides pharmaceutical compositions and isolated host cells, as well as diagnostic and therapeutic kits that comprise at least one MIS receptor agonist or antagonist for use in modulating motor neuron activity in a patient, together with a pharmaceutically acceptable carrier or excipient. Such therapeutic and diagnostic kits may be packaged in suitable container means, such as for commercial distribution, and will also usually additionally comprise instructions for the use of said MIS receptor agonist or antagonist in the particular therapeutic or diagnostic regimen.

The isolated host cells of the invention are preferably mammalian host cells, with human host cells being particularly highly preferred, and may be either isolated, in cell or tissue culture, or even manipulated ex vivo and subsequently introduced into the body of a recipient animal.

In certain embodiments, the creation of non-human host cells, or isolated human host cells that comprise one or more of the disclosed therapeutic compositions is also contemplated to be useful for a variety of diagnostic, and laboratory protocols.

Compositions comprising, consisting essentially of, or consisting of one or more of the disclosed pharmaceutical formulations, or isolated host cells also form part of the present invention, particularly those compositions that further comprise at least a first pharmaceutically-acceptable excipient for use in the manufacture of medicaments and methods involving therapeutic administration of the compounds of the present invention. Such pharmaceutical compositions may optionally further comprise liposomes, a lipid, a lipid complex; or may be comprised within a microsphere or a nanoparticle. Pharmaceutical formulations suitable for intramuscular, intravenous, or direct injection into an organ or tissue or a plurality of cells or tissues of a human or other mammal are particularly preferred.

Other aspects of the invention concern pharmaceutical formulations of the compositions disclosed herein particularly when intended for administration to a mammal through suitable means, such as, by intramuscular, intravenous, or direct injection to cells, tissues, or organs of a selected mammal. Typically, such compositions may be formulated with pharmaceutically-acceptable excipients as described hereinbelow, and may comprise one or more liposomes, lipids, lipid complexes, microspheres or nanoparticle formulations to facilitate administration to the selected organs, tissues, and cells for which therapy is desired.

Kits comprising or consisting essentially of one or more of the disclosed compositions, transformed host cells, or pharmaceutical compositions comprising such; and (ii) instructions for using the kit in a therapeutic, diagnostic, or clinical embodiment also represent preferred aspects of the present disclosure. Such kits may further comprise one or more reagents, buffers, diluents, restriction enzymes, peptides, therapeutics, molecular labels, pharmaceutical compounds, or means for delivery of the preferred compositions to host cells, or to an animal, such as syringes, injectables, and the like. Such kits may be therapeutic kits for treating, preventing, or ameliorating the symptoms of motor neuron diseases, and will typically comprise one or more of the therapeutic compositions described herein, and instructions for using the kit.

### BRIEF DESCRIPTION OF THE DRAWINGS

Further aspects of the present invention will become apparent from the following description which is given by way of example only and with reference to the accompanying drawings, wherein:

FIG. 1A, FIG. 1B, FIG. 1C, FIG. 1D, FIG. 1E, and FIG. 1F show the laser capture microdissection (LCM) of 0.5% cresyl violet acetate stained spinal cord sections. (FIG. 1A to FIG. 1C). The cell bodies of the motor neurons were identified before LCM. (FIG. 1D) The cell bodies of the motor neurons were removed from the sections after LCM. (FIG. 1E) Affixed materials were lifted up with the Cap from the sections after LCM. (FIG. 1F) Less than 3% of GFAP contamination was detected in LCM samples using real-time PCR. *P value<0.001. Scale bar=12.5 μm (FIG. 1A), 25 μm (FIG. 1B) and 50 μm (FIG. 1C, FIG. 1D, and FIG. 1E);

FIG. 2 shows the detection of MISRII transcripts in LCM captured motor neuron samples and different tissues of adult mice by real-time PCR. The target copy numbers were normalized with endogenous GAPDH copy numbers. Values were presented in a log scale and shown as mean±SEM (n=4˜11). The tissues examined were: T, testes; MN, laser-captured motor neurons; SC, spinal cord; B, brain; SN, sciatic nerve; M, skeletal muscle;

FIG. 3A and FIG. 3B show the detection of type II receptors and type I receptors for the TGFβ super family in the LCM captured motor neuron samples using real-time PCR. (FIG. 3A) Five known type II receptors, MISRII, BMPRII, TβRII, ActRIIA and ActRIIB were detected in the spinal motor neurons of adult mice. RET, a receptor of GDNF, was tested as a positive control. (FIG. 3B) Seven known type I receptors, ALK 1 to 7 were detected in the spinal motor neurons of adult mice. Six male and five female adult mice were used in this experiment. Values were shown as mean±SEM. * represented P<0.05 using T-TEST when comparing males to females;

FIG. 4A, FIG. 4B, and FIG. 4C show the detection of MISRII proteins in spinal motor neurons. (FIG. 4A) MISRII proteins were detected in testis (T) and spinal cord (SC) using immunoprecipitation (IP) and Western blot analysis. The positions of the molecular weight markers (in kiloDaltons), MISRII, IgG heavy chain (IgG_{H}) and IgG light chain (IgG_{L}) are indicated. (FIG. 4B) MISRII proteins are localized to motor neurons by immunohistochemistry. The arrow indicates a perinuclear staining of MISRII in the motor neuron. (FIG. 4C) A higher magnification of MISRII staining in the motor neuron using antibody conjugated with Bodipy. Scale bar=50 μm;

FIG. 5 shows a photomicrograph of a section of the lumbar spinal cord of an adult mouse that was stained using an antibody to ALK3. The black arrows point to ALK3 positive motor neurons;

FIG. 6A, FIG. 6B, and FIG. 6C show MIS mRNA in laser-captured motor neurons and other tissues. The bars are the mean±SEM of 11 samples for the motor neurons and four for the other tissues (FIG. 6A) MIS proteins are localized in motor neurons (FIG. 6B) and the granulosa cells of an ovary (FIG. 6C) by immunohistochemistry. The arrows point to a motor neuron in the lumbar spinal cord. MIS staining in granulosa cells (arrowhead) is shown as a positive control. MIS expression is not present in oocyte (arrow). Scale bars=50 μm;

FIG. 7A, FIG. 7B, FIG. 7C and FIG. 7D illustrate cultured motor neurons that have been stained using an antibody to MISRII. The neuron in FIG. 7A was treated with MIS and has multiple highly branched neurites. The neurons in FIG. 7B were from a control culture that lacked MIS. The cell on the top lacks a neurite and may be dead. The cell on the bottom has a single neurite, with no branching; FIG. 7C illustrates the number of differentiated motor neurons (neurons with a neurite) in cultures of motor neurons that have been treated with various doses of MIS for 24 hours. Surviving motor neurons with axons were counted under a phase-contrast microscope; FIG. 7D illustrates the total number of motor neurons in cultures without a growth factor (control, empty bar), 50 ng/ml of MIS or 50 ng/ml of GDNF. Total motor neuron counts of 50 ng/ml MIS or hGDNF treatment after 24 hours. Values are shown as mean±SEM (n=3);

FIG. 8A, FIG. 8B, FIG. 8C, FIG. 8D, FIG. 8E, FIG. 8F, FIG. 8G, and FIG. 8H show the nucleotide sequence (SEQ ID NO:1) of the bovine MIS gene which includes the full length cDNA sequence and the promoter region; the amino acid sequence is shown below the DNA sequence (SEQ ID NO:2) and

FIG. 9A, FIG. 9B, FIG. 9C, FIG. 9D, FIG. 9E, FIG. 9F, FIG. 9G, FIG. 9H, FIG. 9I, and FIG. 9K show the nucleotide sequence (SEQ ID NO:3) of the human MIS gene. The protein sequence (SEQ ID NO:4) is shown below the DNA sequence. It is interrupted in four places by introns.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the inventors' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention is directed to the surprising finding that MIS and MIS receptors are present in motor neurons and that an MIS receptor agonist or antagonist is effective at modulating motor neuron survival.

The present invention therefor provides methods of modulating motor neuron activity and/or motor neuron survival in a patient by administering to said patient an effective amount of an agonist or antagonist of one or more MIS receptors.

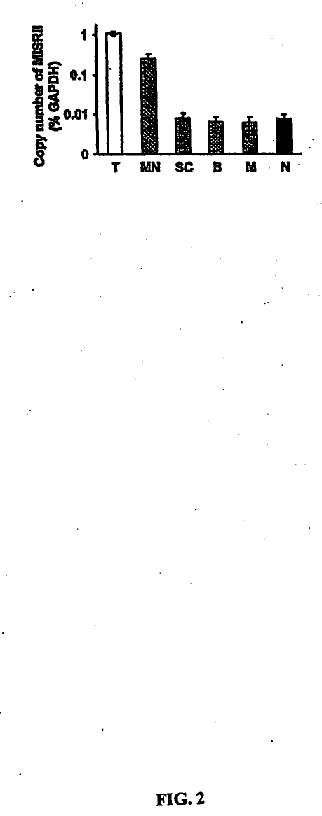

In work leading up to the present invention, mRNA was extracted from isolated motor neurons and used to investigate differential gene expression in motor neurons. This work lead to the identification of MIS receptor MISRII in motor neurons, as shown in FIG. 2 to FIG. 4.

Müllerian inhibitory substance (MIS) is a member of the TGFβ superfamily and growth factors whose functions were believed to be restricted to reproductive tissues.

TGFβ superfamily members signal through a heteromeric receptor complex, containing a type I and type II receptor. Type II Müllerian inhibitory substance receptor (MISRII) has been identified and considered to be the only type II receptor for MIS. However, the identification of a type I receptor for MIS has been controversial, with a number of different type I receptors being suggested to mediate MIS signals. Suggested receptors include ALK-2, ALK-3 and ALK-6.

Unexpectedly, the inventors have now identified that MIS is present in motor neurons. In particular, the colocalisation of MIS and MISRII in motor neurons is consistent with MIS being an autocrine regulator of motor neuron function, or a paracrine regulator of motor neuron to motor neuron interactions.

The terms “Müllerian inhibitory substance” and “anti-Müllerian hormone” are synonymous and can be used interchangeably. The equivalent terms when used in relation to their respective receptors are to be similarly understood.

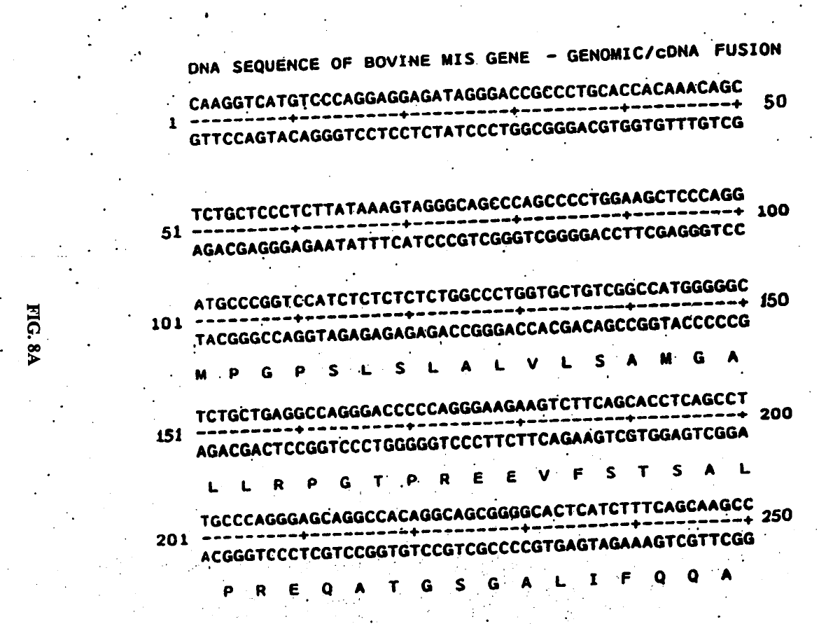

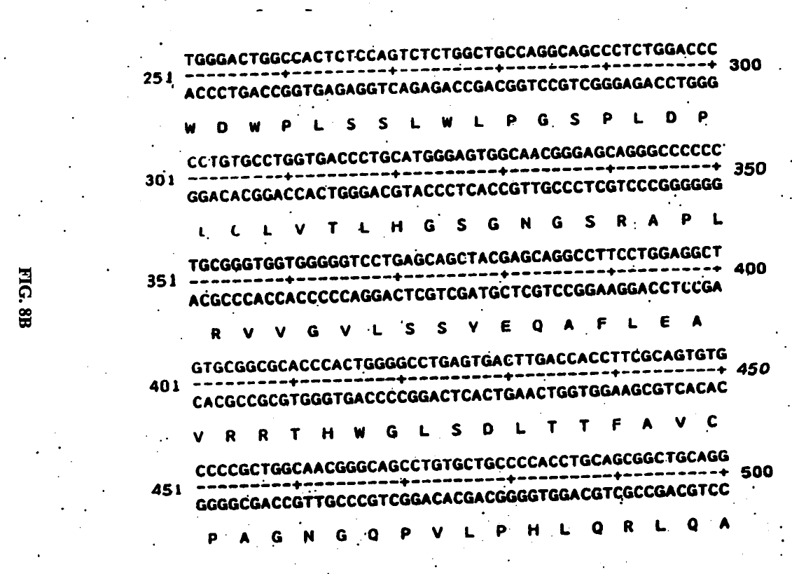

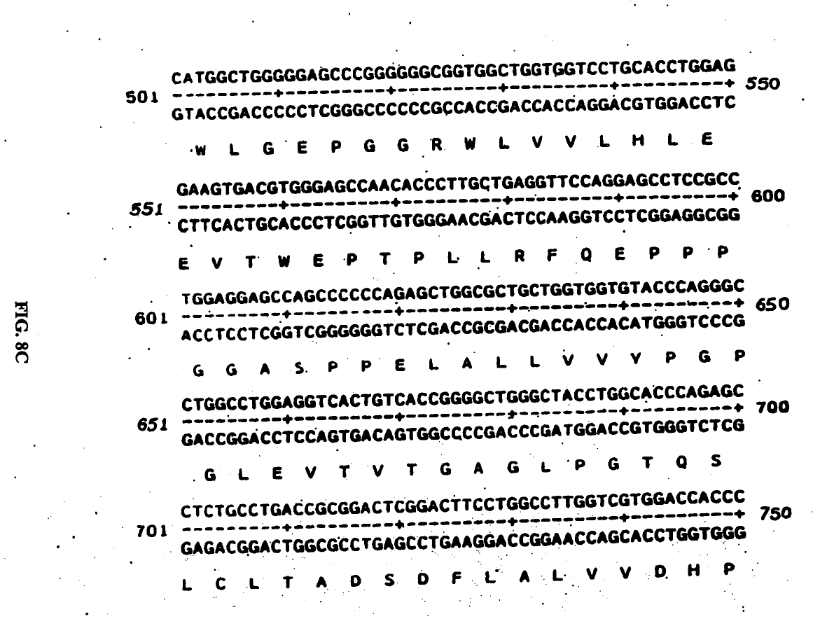

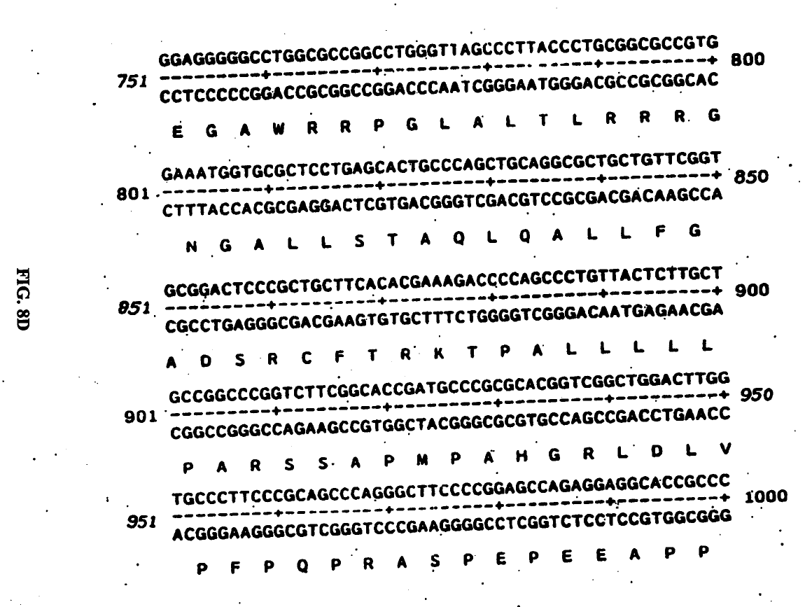

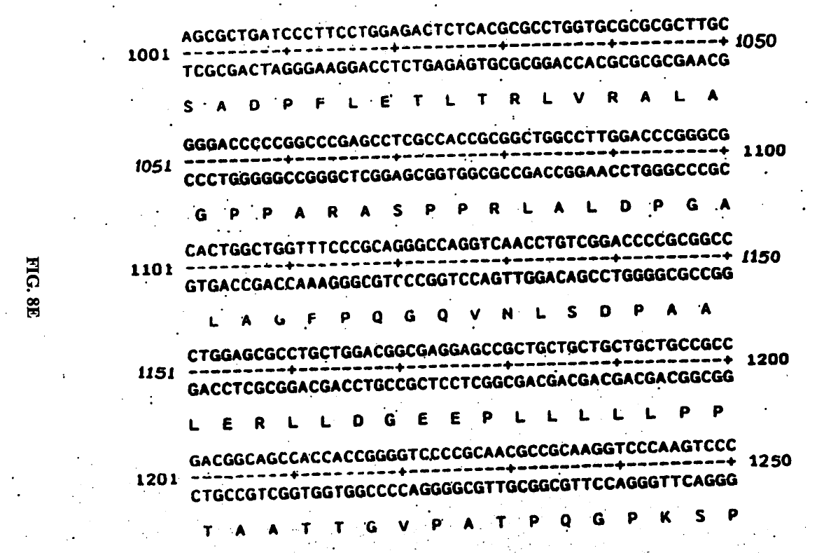

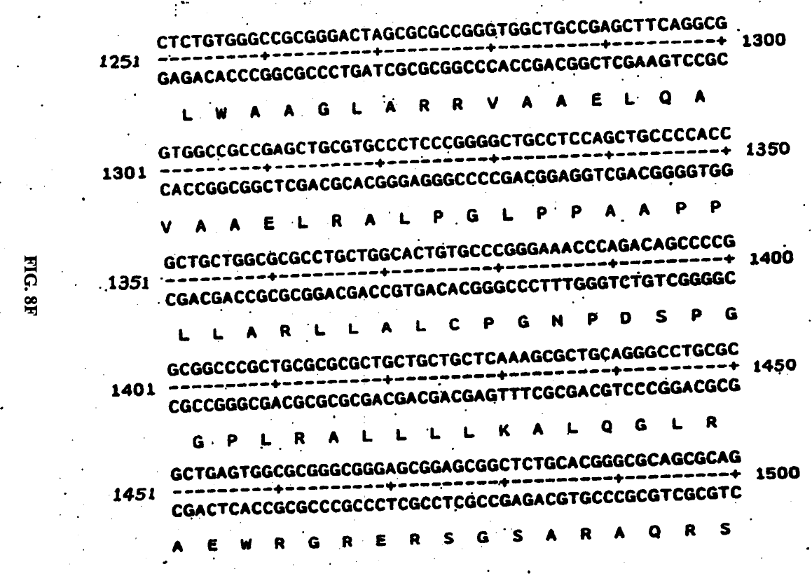

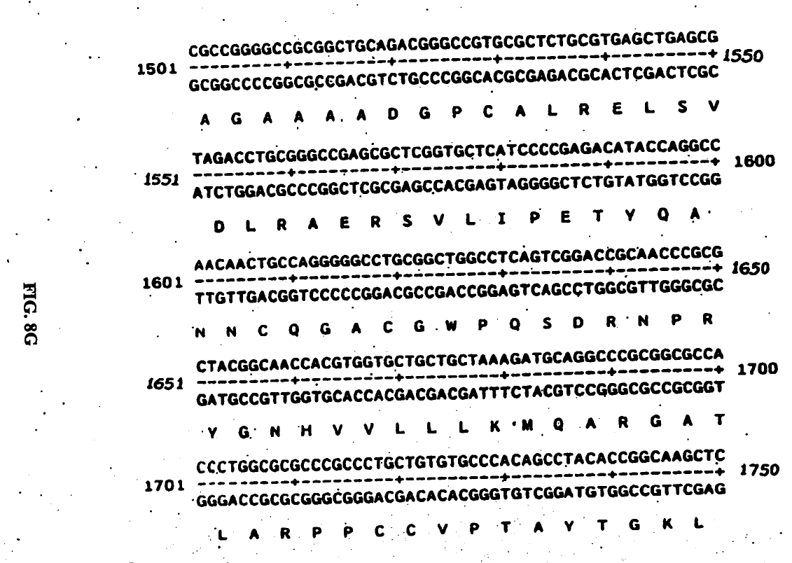

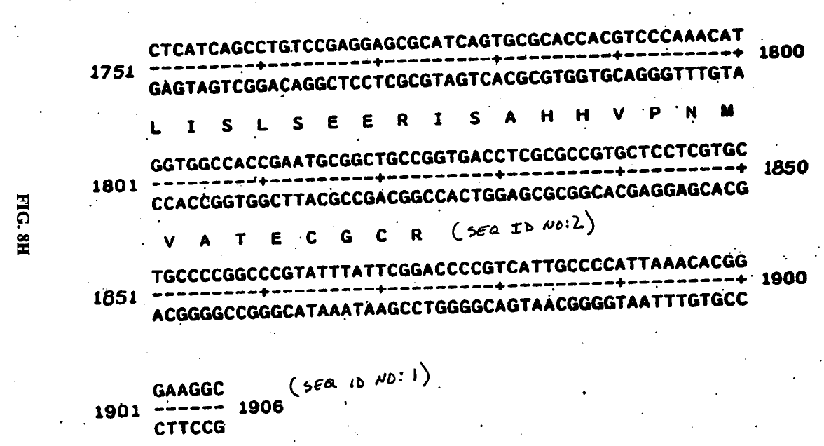

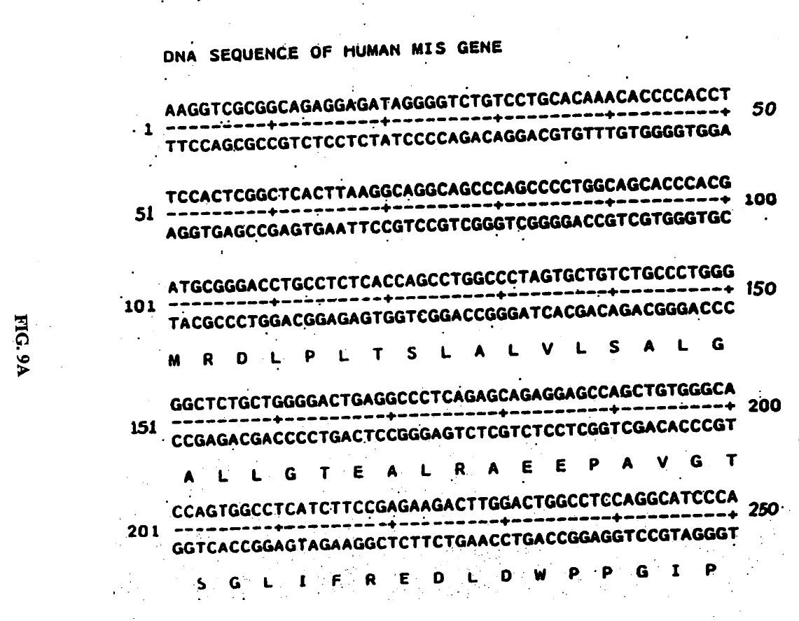

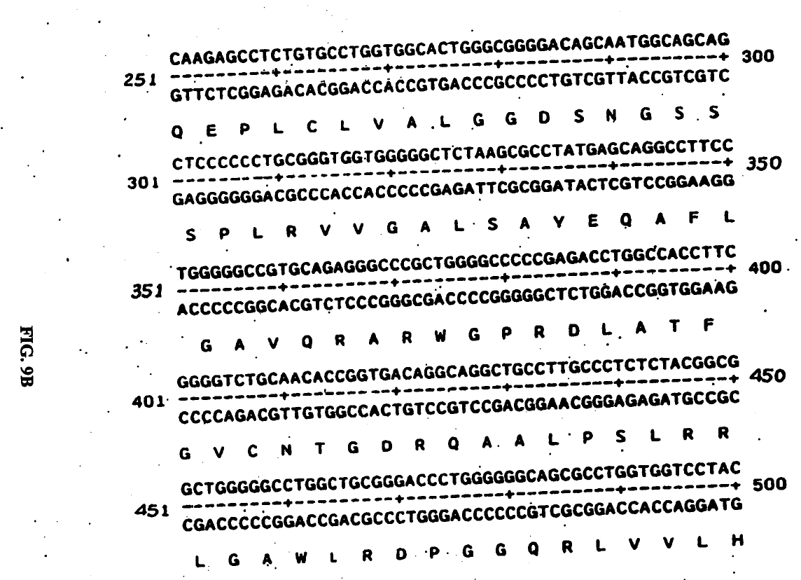

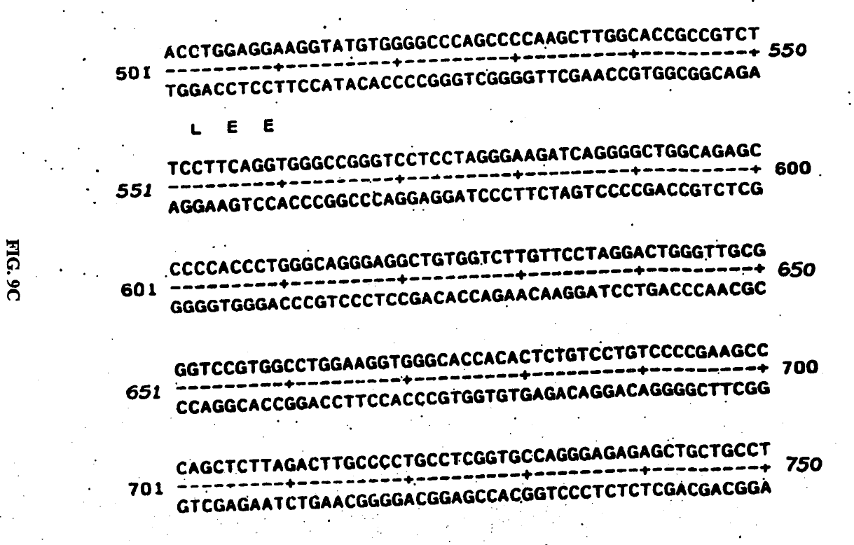

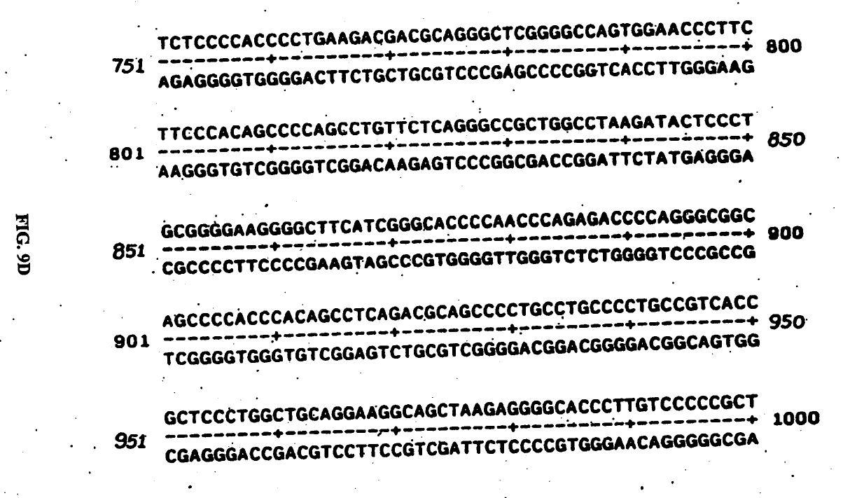

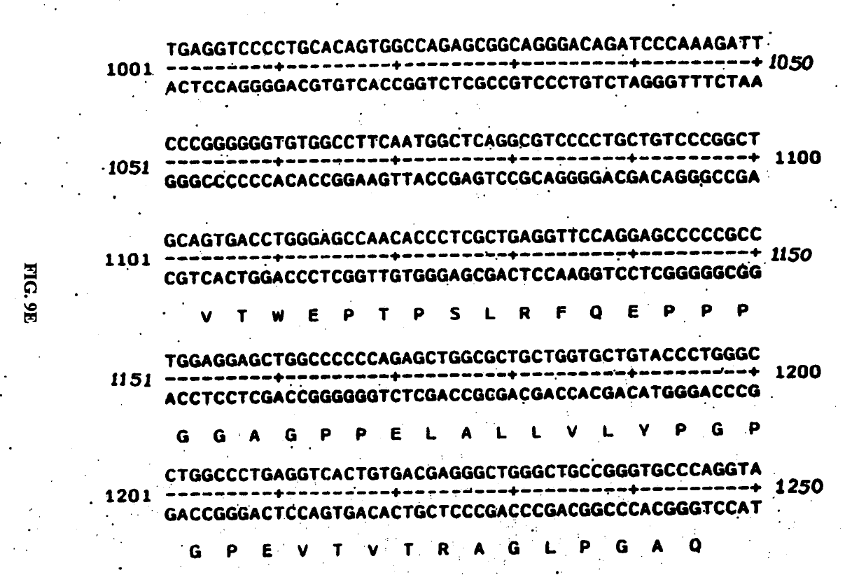

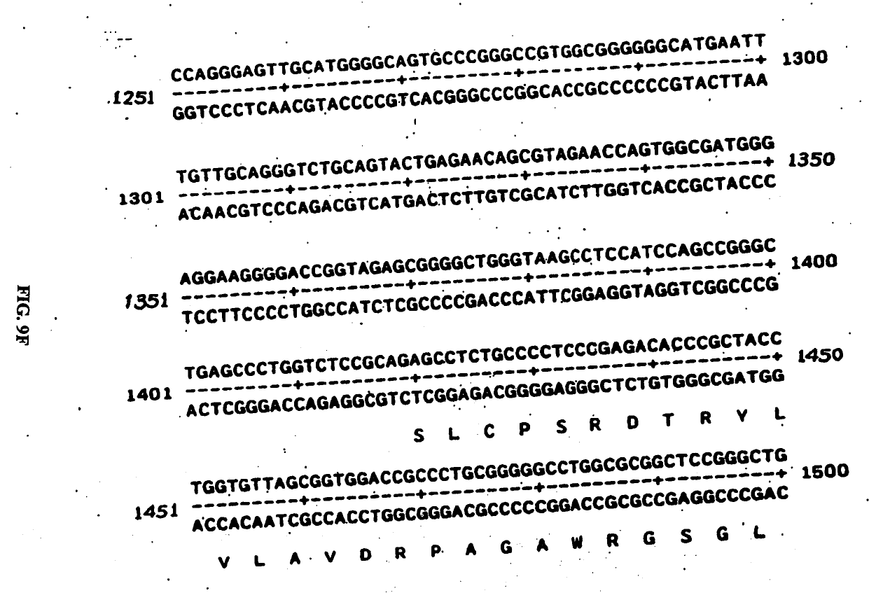

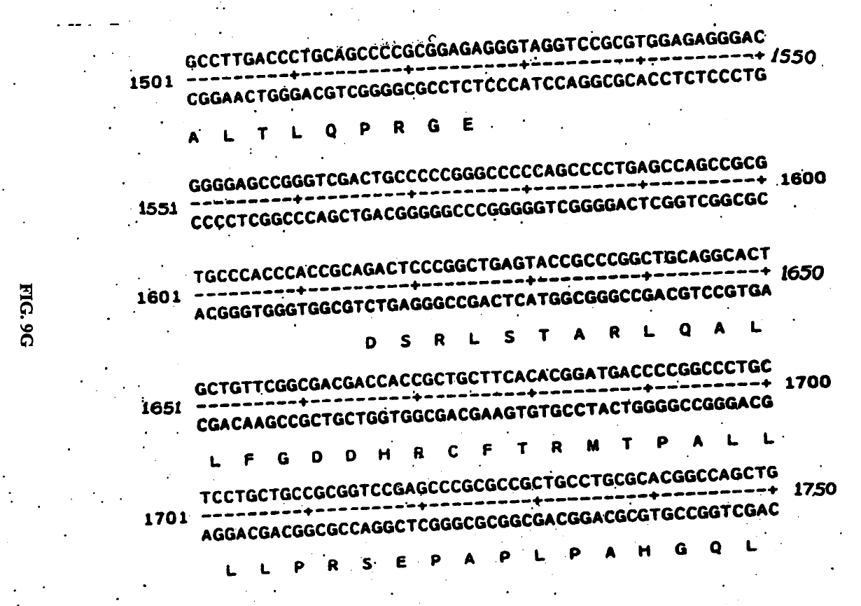

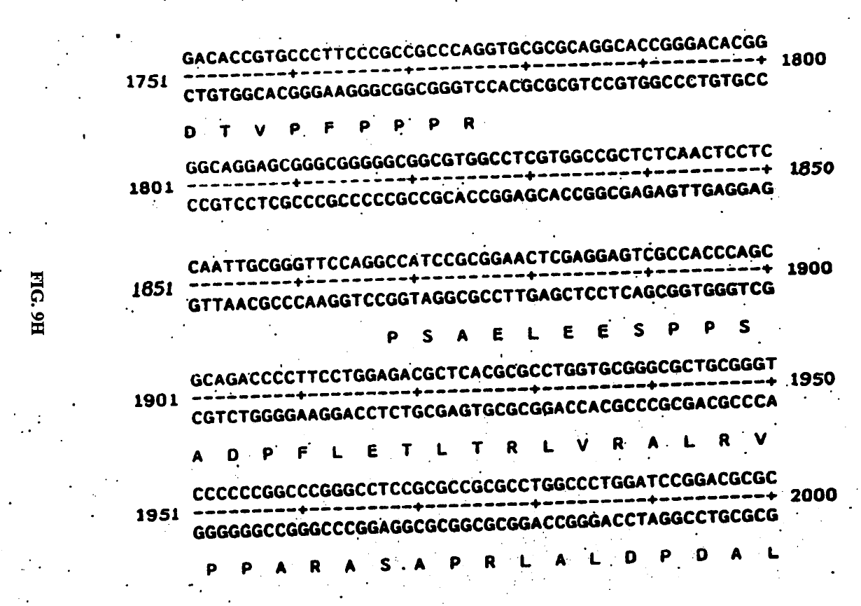

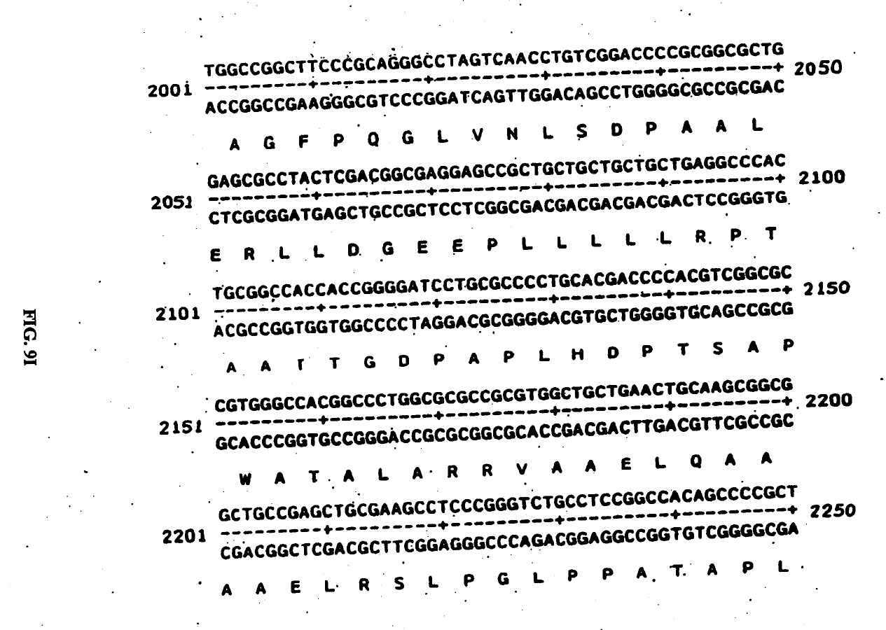

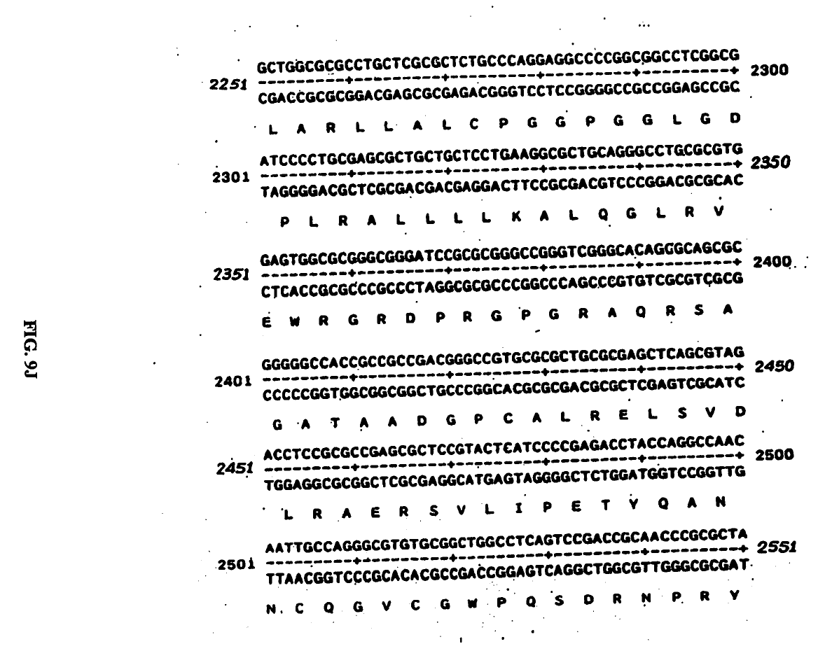

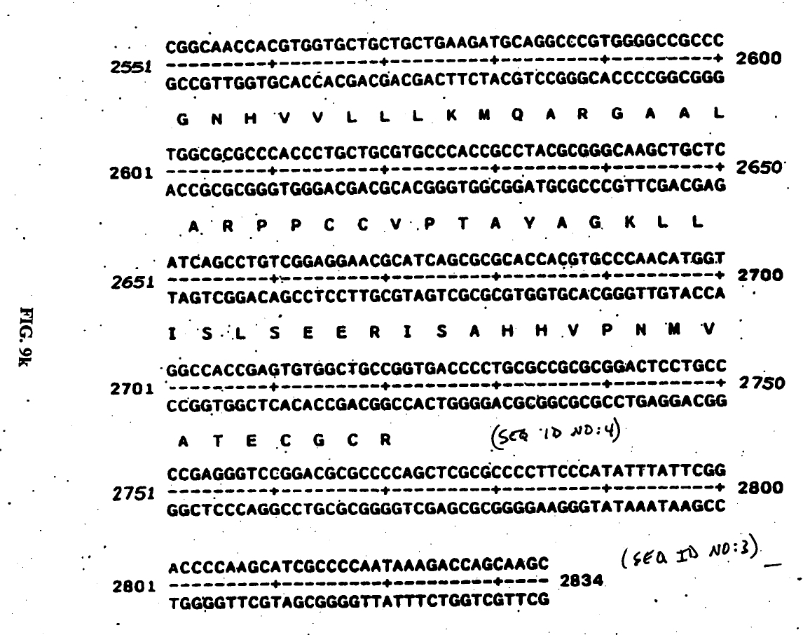

FIG. 8 shows the nucleotide (SEQ ID NO: 1) and amino acid (SEQ ID NO:2) sequences of bovine MIS (GenBank Accession Number M13151). FIG. 9 shows the nucleotide (SEQ ID NO:3) and amino acid (SEQ ID NO:4) sequences of human MIS (GenBank Accession Number K03474). Both amino acid sequences set forth in SEQ ID NO:2 and SEQ ID NO:4 include a 24 amino acid leader peptide as amino acids 1 to 24.

The complete nucleotide sequences for bovine and human MIS are also disclosed in U.S. Pat. No. 5,047,336 (which is specifically incorporated herein by reference in its entirety). A comparison of the amino acid sequence for human and bovine MIS is shown in Cate et al., Handbook of Experimental Pharmacology 95/11:184, edited by M. B. Sporn and A. B. Roberts, Spinger-Verlag Berlin Heidelberg (1990).

Endogenous MIS has been shown to be produced as a 140-kDa glycosylated disulfide-linked homodimer (Pepinsky et al., J. Biol. Chem., 263:18961-4 [1988]). Under reducing conditions, the protein migrates on gel electrophoresis at an apparent molecular weight of 70 kDa. The protein can be proteolytically cleaved into two distinct fragments: a transforming growth factor (TGF)-β-like C-terminal fragment that migrates electrophoretically as a 12.5-kDa fragment; and a N-terminal fragment that migrates electrophoretically as a 57-kDa fragment (Pepinsky et al., J. Biol. Chem., 263:18961-4 [1988]).

The term “carboxy-terminal (C-terminal) fragment of MIS” is intended to include compounds and materials structurally similar to the about 12.5 kDa (about 25 kDa under non-reducing conditions) C-terminal fragment of MIS and of about 109 amino acids in length, resulting from proteolytic (e.g., plasmin) cleavage at residue 427 of the 535 amino acid human MIS monomer (residue 451 of SEQ ID NO:4) and the proteolytic (e.g., plasmin) cleavage site is at residue 443 of the 551 amino acid bovine MIS molecule (residue 466 of SEQ ID NO:2). In particular, “carboxy-terminal (C-terminal) fragment of MIS” is intended to include the about 25 kDa homodimeric C-terminal fragment of MIS. The plasmin digested MIS has been shown to remain fully active in the organ culture assay (Pepinsky et al., J. Biol. Chem., 263:18961-4 [1988]).

The C-terminal amino acid and nucleotide sequences for bovine MIS and human MIS are shown in FIGS. 17 and 18, respectively, of U.S. Pat. No. 5,661,126 (which is specifically incorporated herein by reference in its entirety).

The term “modulating motor neuron activity” should be taken to mean the artificial interference on motor neuron activity. This may include treatments that either increase or decrease total motor neuron cell number; or that either increase or decrease motor neuron receptor signal transduction to the cell, the sensitivity or total number of motor neuron receptors; when compared to the endogenous levels observed in a patient in need thereof prior to treatment.

The term “enhancing motor neuron survival” should be taken to mean treatments which increase or preservation of neuron differentiation or total neuron number compared to that observed in a patient in need thereof prior to treatment or in an untreated sample of an in-vitro culture.

The term “patient” as used herein is preferably a mammal and includes humans, and non-human mammals such as cats, dogs, horses, cows, sheep, pigs, rabbits, deer, mice, possum, and others, including for examples, ungulates, porcines, ovines, bovines, equines, felines, canines, lupines, caprines, rodents, and primates. Such patients include for example, human patients under medical care, or pets, domesticated farm animals, livestock, and/or zoological animals under veterinary medical care. In illustrative embodiments, the patient is a mammal, and more particularly, human.

The term “comprising” as used in this specification and the claims is consistent with long-standing U.S. patent practice, and is considered an “open” term that means “containing at least” or “consisting at least in part of”. When interpreting statements in this specification and the claims which include that term, the features, prefaced by that term in each statement or claim, all need to be present but other features can also be present.

The term “agonist or antagonist” means a biologically-active agent that is capable of modulating a biological process. Agonists will usually be interpreted as inducing an augmentation of the biological process. Antagonists will usually be interpreted as inducing an inhibition of a biological process.

In this context agonists include ligands that will bind to MIS, the C-terminal fragment of MIS, or a MIS receptor and enhance the signal transduction to the cell, the sensitivity or number of receptors. Examples of such ligands include MIS, a functional derivative or analog thereof, a polypeptide or an antibody or antibody fragment raised against MIS, the C-terminal fragment of MIS, or a MIS receptor or portions thereof.

In accordance with long standing patent law convention, the words “a” and “an” when used in this application, including the claims, denotes “one or more”.

In one embodiment MIS or functional derivatives thereof with the ability to bind to a MIS receptor may be provided as agonists to patients in order to enhance motor neuron activity and/or survival in accordance with a method of the present invention.

In this context antagonists include ligands that will bind to MIS, the C-terminal fragment of MIS, or a MIS receptor and decrease the signal transduction to the cell, the sensitivity or number of receptors. Examples of such ligands include antibodies or fragments thereof capable of binding to MIS, variants of MIS having the ability to bind to MIS receptor molecules but lacking other MIS activity, a MIS receptor molecule, and variants of a MIS receptor molecule which have the ability to bind to MIS.

In one embodiment antagonists of MIS, the C-terminal fragment of MIS, or a MIS receptor may be provided to patients in order to decrease motor neuron activity in accordance with a method of the present invention, decreasing a patient's response to the presence of MIS.

Other agonists or antagonists include molecules that will affect the bioavailability of endogenous ligands. Such molecules include purified or recombinant MIS receptors or fragments thereof that will bind or scavenge endogenous ligands that naturally bind to MIS receptors in vivo, thereby modulating the activity of the MIS regulatory pathway.

The MISRII gene has been isolated in the rat (Baarends, W. M. et al., [1994] Development, 120:189-197), rabbit (di Clemente, N. et al., [1994] Mol. Endocrinol., 8:1006-1020), human (Imbeaud, S. et al., [1995] Nat. Genet., 11:382-388) and mouse (Mishina, Y. et al., [1996] Genes Dev., 10:2577-2587). The complete coding sequence and mRNA of the mouse MISRII gene is available as GenBank Accession Numbers AF503863 and NM_{—}144547 respectively. The complete coding sequence and mRNA of the human MISRII gene are available as GenBank Accession Numbers AF172932 and NM_{—}020547, respectively.

In one embodiment the agonist or antagonist may be a recombinant protein comprising a MIS receptor binding or ecto-domain. Exon 2 of the MISRII gene seems to be essential for ligand binding, as shown in the rabbit receptor (di Clemente, N. et al., [1994] Mol. Endocrinol., 8:1006-1020).

The term “Müllerian inhibitory substance” is intended to include compounds and materials which are structurally similar to MIS. Examples of such included substances and materials are salts and functional derivatives of MIS. Additionally, the present invention is intended to include variants of MIS which have substantially the same biological activity as MIS. Examples of such variants include MIS molecules carrying a deletion, insertion, or alteration in the amino acid sequence. MIS may be obtained from any mammalian source or from non-mammalian sources through the use of recombinant DNA technology, or from the chemical synthesis of the MIS protein.

A “functional derivative” of MIS is a compound which possesses biological activity (either functional or structural) that is substantially similar to the biological activity of MIS. The term “functional derivatives” is intended to include the “fragments,” “variants”, “analogs” or “chemical derivatives” of MIS.

A “fragment” of a molecule such as MIS refers to any polypeptide subset of the molecule. Fragments of MIS which have activity and which are soluble (i.e., not membrane-bound) are especially preferred.

One preferred fragment is the transforming growth factor (TGF)-β-like C-terminal fragment of MIS of about 109 amino acids in length that is formed by the proteolytic (e.g., plasmin) cleavage of MIS and which migrates electrophoretically as a 12.5-kDa fragment (Pepinsky et al., J. Biol. Chem., 263:18961-4 [1988]). The Müllerian duct regression and antiproliferative bioactivities of MIS have previously been shown to reside in the C-terminal domain of MIS as described in U.S. Pat. No. 5,661,126, which is specifically incorporated herein by reference in its entirety.

A “variant” of a molecule such as MIS is meant to refer to a molecule substantially similar in structure and function to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants and that term is used herein even if the structure of one of the molecules not found in the other, or if the sequence of amino acid residues is not identical.

The term “analog” refers to a compound substantially similar in function to either the entire molecule or to a fragment thereof.

As used herein, a molecule is said to be a “chemical derivative” of another molecule when it contains additional chemical moieties not normally a part of the molecule. Such moieties may impart a biological function with improved characteristics over the native compound (e.g., such an analog may have a longer half-life than the native compound). The moieties may alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990).

Sequence comparison of functional derivatives may be achieved using BLASTP.

More particularly, polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI via their ftp internet server (ftp://ftp.ncbi.nih.gov/blast/). The default parameters of bl2seq may be utilised.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X., [1994] On Global Sequence Alignment. Computer Applications in the Biosciences 10:227-235) are also suitable global sequence alignment programs for calculating polypeptide sequence identity.

Use of BLASTP as described above is preferred for use in the determination of polypeptide variants useful in the present invention.

Variant polynucleotide and polypeptide sequences refer to polynucleotide or polypeptide sequences different from the specifically identified MIS sequences, wherein one or more nucleotides or amino acid residues is deleted, substituted or added. Variants may be naturally occurring allelic variants, or non-naturally occurring variants. Variants may be from the same or other species and encompasses homologues, paralogues and orthologues. Both cDNA and genomic sequence variants are contemplated. Variant sequences preferably exhibit at least 70%, at least 80%, preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a MIS sequence useful in the present invention. For polypeptides, identity is found over a comparison window of at least 15, preferably at least 18 amino acid positions, more preferably at least 20 amino acid positions, and most preferably over the entire length of a polypeptide.

Sequence identity may be determined as discussed above.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science, 247:1306) and PCT Intl. Appl. Publ. No. WO 01/27143.

The variant sequences, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucl. Acids Res., 29:1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

The term “substantially complementary,” when used to define either amino acid or nucleic acid sequences, means that a particular subject sequence, for example, a nucleotide sequence, is substantially complementary to all or a portion of the selected sequence, and thus will specifically bind to a portion of an mRNA that encodes the selected sequence. Typically sequences that are substantially complementary will be sequences that will have no more than about 1, 2, 3, 4, 5, 6, 7, 8, 9, or 10 base mismatches throughout the complementary portion of the sequence. In many instances, it may be desirable for the sequences to be exact matches, i.e. be completely complementary to the sequence to which a nucleotide segment specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleotide sequences typically will be greater than about 80 percent complementary (or ‘% exact-match’) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to all or a portion of the target mRNA to which the designed oligonucleotide specifically binds.

An exemplary family of programs useful for identifying variants in sequence databases is the BLAST suite of programs (version 2.2.10 [October 2004]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA.

The use of the BLAST family of algorithms, including BLASTN, BLASTP, and BLASTX, is described in the publication of Altschul et al., Nucleic Acids Res. 25:3389-3402, 1997.

Multiple sequence alignments of a group of related sequences can be carried out with CLUSTALW (Thompson, J. D., Higgins, D. G. and Gibson, T. J. [1994] Nucl. Acids Res., 22:4673-4680, http://www-igbmc.u-strasbg.fr/BioInfo/ClustalW/Top.html) or T-COFFEE (Cedric Notredame, Desmond G. Higgins, Jaap Heringa, T-Coffee, J. Mol. Biol., [2000] 302:205-217) or PILEUP, which uses progressive, pairwise alignments. (Feng and Doolittle, [1987] J. Mol. Evol., 25:351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, [1994] Nulc. Acids Res., 22:3583; Hofmann et al., [1999] Nucl. Acids Res., 27:215) is a method of identifying the functions of uncharacterised proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., [2002] Nucl. Acids Res., 30:235). Prosearch is a tool that enables a user to search a number of databases including SWISS-PROT, SWISS-2DPAGE, SWISS-3DIMAGE, and ENZYME, as well as other cross-referenced databases such as EMBL, GenBank, OMIM, Medline databases etc with a given sequence pattern or signature.

In addition to the computer/database methods described above, polypeptide variants may be identified by physical methods, for example by screening expression libraries using antibodies raised against MIS polypeptides used in the invention (Sambrook et al., Molecular Cloning: A Laboratory Manual, 2nd Ed. Cold Spring Harbor Press, 1987) or by identifying polypeptides from natural sources with the aid of such antibodies.

Variant polynucleotides useful herein also or alternately hybridize to the polynucleotide sequences recited above, or complements thereof, antisense sequences and complements thereof, under stringent conditions. As used herein, “stringent conditions” refers to hybridization conditions such as pre-washing in a solution of 6×SSC, 0.2% SDS; hybridizing at 65° C., 6×SSC, 0.2 SDS overnight; followed by two washes of 30 minutes each in 1×SSC, 0.1% SDS at 65° C. and two washes of 30 minutes each in 0.2×SSC, 0.1% SDS at 65° C. Such conditions are discussed more fully in, for example, Sambrook et al., Molecular Cloning, supra.

The receptor-ligand interaction may be determined using the yeast hybrid system, which involves the expression of chimeric proteins and their subsequent interaction within the yeast cell nucleus (Topcu and Dorden [2000] Pharm. Res 17, 9). Unlike in-vitro biochemical techniques such as immunoprecipitation, the yeast hybrid system can detect in-vivo interactions, and does not require protein purification or antibody production. Accordingly, the yeast hybrid system is a powerful technique for identifying protein-protein interactions.

The term “antibody or antibody fragment” means an intact molecule or fragment thereof, which are capable of binding an antigenic determinant of MIS, the C-terminal fragment of MIS, or a MIS receptor (i.e., that portion of a molecule (i.e., an epitope) that makes contact with a particular antibody or other binding molecule). Antibodies include, for example, polyclonal, monoclonal, chimeric, and single chain antibodies, single chain Fvs, Fab fragments, and fragments produced by a Fab expression library, or phage display. The antibodies of the present invention may also be produced by genetic engineering methods such as chimeric and humanised monoclonal antibodies, comprising both human and non-human portions, which can be made using standard recombinant DNA techniques.

In one embodiment the antibody fragment may be Fab, F(ab′)2, Fv or single chain Fv (scFv), in which Fv fragments from H and L chains are ligated by an appropriate linker (Huston et al., [1988] Proc. Natl. Acad. Sci. USA, 85:5879-83). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, e.g., Co et al., [1994] J. Immunol., 152:2968-76; Better M. and Horwitz [1989] Methods Enzymol., 178:476-96; Pluckthun and Skerra [1989] Methods Enzymol., 178:497-515; Lanioyi [1986] Methods Enzymol., 121:652-63; Rousseaux et al., [1986] Methods Enzymol., 121:663-9; and Bird and Walker [1991] Trends Biotechnol., 9:132-7).

An antibody may be modified by conjugation with a variety of molecules, such as polyethylene glycol (PEG). The modified antibody can be obtained by chemically modifying an antibody. These modification methods are conventional in the field.

In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a MIS or MIS receptor polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL TDM adjuvant (moriophosphoryl Lipid A, synthetic trehalose dicorynomycolate). The immunization protocol may be selected by one skilled in the art without undue experimentation.

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the MIS, the C-terminal fragment of MIS, or MIS receptor polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [see e.g., Goding, Monoclonal Antibodies: Principles and Practice Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), that will prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (J. Immunol., 133:3001 [1984]; Brodeur et al, Monoclonal Antibody Production Techniques and Applications, Marcel Dekker, Inc., New York, [1987] pp. 51-63).

The culture medium in which the hybridoma cells are cultured can then be assayed for the presence of monoclonal antibodies directed against the MIS, the C-terminal fragment of MIS, or MIS receptor polypeptide. Preferably, the binding specificity of monoclonal antibodies produced by the hybridoma cells is determined by immunoprecipitation or by an in vitro binding assay, such as radio linked immunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (see e.g, Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxyapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies. The hybridoma cells of the invention serve as a preferred source of such DNA. Once isolated, the DNA may be placed into expression vectors, which are then transfected into host cells such as simian COS cell Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 [1986]; Riechmann et al., Nature, 332:323-329 [1988]; and Presta, Curr. Op. Struct. Biol. a:593-596 [1992]).

Methods for humanizing non-human antibodies are well known in the art. Generally, a humanized antibody has one or more amino acid residues introduced into it from a source which is non human. These non-human amino acid residues are often referred to as “import’ residues, which are typically taken from an “import” variable domain. Humanization can be essentially performed following the method of Winter and co-workers (Jones et al., Nature, 321:522-525 [1986]; Riechmann et al., Nature, 332:323-327 [1988]; Verhoeyen et al., Science, 239:1534-1536 [1988]), by substituting rodent CDRs or CDR sequences for the corresponding sequences of a human antibody. Accordingly, such “humanized” antibodies are chimeric antibodies (see e.g., U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 [1991]; Marks et al., J. Mol Biol., 222:581 [1991]). The techniques of Cole et al. and Boerner et al. are also available for the preparation of human monoclonal antibodies (Cole et al., Monoclonal Antibodies and Cancer Therapy Alan R. Liss, p. 77 [1985] and Boerner et al., J. Immunol., 147(1):86-95 [1991]). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10:779-783 (1992); Lonberg et al., Nature, 368:856-859 (1994); Morrison, Nature, 368:812-13 (1994); Fishwild et al., Nature Biotechnol., 14:845-51 (1996); Neuberger, Nature Biotechnol., 14:826 (1996); Lonberg and Huszar, Intern. Rev. Immunol., 13:65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

Bispecific antibodies are monoclonal, preferably human or humanized, antibodies that have binding specificities for at least two different antigens. In the present case, one of the binding specificities is for MISRII, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy chain light-chain pairs, where the two heavy chains have different specificities (Milstein and Cuello, Nature, 305:537-539 [1983]). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in PCT Intl. AppI. Publ. No. WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods Enzymol., 121:210 (1986).

According to another approach described in PCT Intl. Appl. Publ. No. WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to forrn the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med., 175:217-225 (1992) describe the production of a fully humanized bispecific antibody F(ab′) molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol, 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Nati. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol, 152:5368 (1994).

Antibodies with more than two valencies are contemplated. For example, trispecific antibodies can be prepared. Tutt et al., J. Immunol., 147:60 (1991).

Exemplary bispecific antibodies may bind to two different epitopes on a given MIS receptor polypeptide herein. Alternatively, an anti-MIS receptor polypeptide arm may be combined with an arm which binds to a triggering molecule on a leukocyte such as a T-ceil receptor molecule (e.g., CD2, CD3, CD28, or B7), or Fc receptors for IgG (FcγR), such as FcγRI (CD64), FcγRII (CD32) and FcγRII (CD 16) so as to focus cellular defense mechanisms to the cell expressing the particular MIS receptor polypeptide. Bispecific antibodies may also be used to localize cytotoxic agents to cells which express a particular MIS receptor polypeptide. These antibodies possess a MISR-binding arm and an arm which binds a cytotoxic agent or a radionuclide chelator, such as EOTUBE, DPTA, DOTA, or TETA. Another bispecific antibody of interest binds the MIS receptor polypeptide and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT Intl. Appl. Publ. No. WO 91/00360; PCT Intl. Appl. Publ. No. WO 92/200373). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include immunothiolate and methyl-4-mercaptobutyriniidate and those disclosed, for example, in U.S. Pat. No. 4,676,980.

It may be desirable to modify the antibody of the invention with respect to effector function, so as to enhance, e.g., the effectiveness of the antibody. For example, cysteine residue(s) may be introduced into the Fe region, thereby allowing interchain disulfide bond formation in this region. The homodimeric antibody thus generated may have improved internalization capability and/or increased complement-mediated cell killing and antibody-dependent cellular cytotoxicity (ADCC). See Caron et at., J. Exp Med., 176:1191-1195 (1992) and Shopes, J. Immunol., 148:2918-2922 (1992). Homodimeric antibodies may also be prepared using heterobifunctional cross-linkers as described in Wolff et cii. Cancer Res, 53:2560-2565 (1993). Alternatively, an antibody can be engineered that has dual Fe regions and may thereby have enhanced complement lysis and ADCC capabilities. See Stevenson et al., Anti-Cancer Drug Design, 3:219-230 (1989).

The methods of the invention may also comprise altering the expression level or activity of MIS, the C-terminal fragment of MIS, or a MIS receptor in the patient. This may be by promoting expression, or administration of composition comprising a polynucleotide coding for a Müllerian inhibitory substance receptor agonist or antagonist. Alternatively, this may be by inhibiting expression. Whether promotion or inhibition of expression levels is appropriate will depend on the desired effect. Without wishing to be bound by theory, both over- and under-expression of polynucleotides are believed to be possible at this time.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods of the invention.

The term “antisense-oligonucleotides” as used herein encompasses both nucleotides that are entirely complementary to the target sequence and those having a mismatch of one or more nucleotides, so long as the antisense-oligonucleotides can specifically hybridize to the target sequence. For example, the antisense-oligonucleotides of the present invention include polynucleotides that have an identity of at least 70% or higher, preferably at 80% or higher, more preferably 90% or higher, even more preferably 95% or higher over a span of at least 15 contiguous nucleotides to either of the nucleotide sequences set forth in SEQ ID NO:1 and SEQ ID NO:3.

Algorithms known in the art as discussed above can be used to determine the identity. Furthermore, derivatives or modified products of the antisense-oligonucleotides can also be used as antisense-oligonucleotides in the present invention. Examples of such modified products include lower alkyl phosphonate modifications such as methyl-phosphonate-type or ethyl-phosphonate-type, phosphorothioate modifications and phosphoroamidate modifications.

Antisense-oligonucleotides corresponding to the nucleotide sequence of MIS, the C-terminal fragment of MIS, or a MIS receptor can be used to reduce expression in situations where that is required. The anti-sense-oligonucleotides of the present invention may act by binding to the polypeptides coding for MIS, the C-terminal fragment of MIS, or a MIS receptor or mRNAs corresponding thereto and thereby inhibiting the transcription or translation thereof, promoting the degradation of the mRNAs, and/or inhibiting the expression of the proteins encoded by the nucleotides, and finally inhibiting the function of the proteins.

In one embodiment expression may be inhibited by administering an antisense composition to the patient, the composition comprising a polynucleotide sequence complementary to SEQ ID NO:1 or SEQ ID NO:3.

The nucleic acids that inhibit one or more gene products also include small interfering RNAs (siRNA) comprising a combination of a sense strand nucleic acid and an antisense strand nucleic acid of a nucleotide sequence coding for MIS, the C-terminal fragment of MIS, or a MIS receptor. The term “siRNA” refers to a double stranded RNA molecule which prevents translation of a target mRNA. Standard techniques of introducing siRNA into the cell can be used in the treatment or prevention of the present invention, including those in which DNA is a template from which RNA is transcribed. The siRNA is constructed such that a single transcript has both the sense and complementary antisense sequences from the target gene, e.g., a hairpin.

The nucleotide sequence of siRNAs may be designed using a siRNA design computer program available from the Ambion website (http:/Iwww.ambion.com/techlib/misc/siRNA_finder.html). Nucleotide sequences for the siRNA are selected by the computer program based on the following protocol:

Selection of siRNA Target Sites:

1. Beginning with the AUG start codon of transcript, scan downstream for AA dinucleotide sequences. Record the occurrence of each AA and the 3′ adjacent 19 nucleotides as potential siRNA target sites. Tusehi, et al. recommend not to design siRNA against the 5′ and 3′ untranslated regions (UTRs) and regions near the start codon (within 75 bases) as these may be richer in regulatory protein binding sites, and thus the complex of endonuclease and siRNAs that were designed against these regions may interfere with the binding of UTR-binding proteins and/or translation initiation complexes.

2. Compare the potential target sites to the human genome database and eliminate from consideration any target sequences with significant homology to other coding sequences. The homology search can be performed using BLAST, which can be found on the NCBI server at: www.ncbi.nlm.nih.govIBLAST/

3. Select qualifying target sequences for synthesis. On the website of Ambion, several preferable target sequences can be selected along the length of the gene for evaluation.

The siRNAs inhibit the expression of MIS, the C-terminal fragment of MIS, or a MIS receptor protein and is thereby useful for suppressing the biological activity of proteins. In one embodiment expression is inhibited by administering a siRNA composition to the patient the composition reducing the expression of a sequence set forth in SEQ ID NO: 1 or SEQ ID NO:3.

It is contemplated that the agonists or antagonists of the invention will be tested for biological activity in an animal model or in an in vitro model and suitably active compounds formulated into pharmaceutical compositions.

Recombinant MIS can be expressed in a protein expression system. The use of prokaryotic and eukaryotic expression systems are well known to those in the art. For example, bacterial (e.g., E. coli), fungi (e.g., yeast), mammalian cells (e.g., CHO cells, COS cells) plant cells or insect cells (e.g., baculovirus transformed cells) expression systems can be used. U.S. Pat. No. 5,047,336 describes recombinant DNA techniques which can readily be used to produce MIS or a fragment thereof.

Methods for purifying non-recombinant MIS are also well known in the art and have been described in U.S. Pat. Nos. 4,404,188; 4,487,833; and 5,011,687.

Therapeutic formulations preferably containing agonists or antagonists of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Compounds identified by the screening assays disclosed herein can be formulated in an analogous manner, using standard techniques well known in the art.

The formulations to be used for in vivo administration must be sterile. This is readily accomplished by filtration through sterile filtration membranes.

Lipofections or liposomes can also be used to deliver the compounds into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Nat. Acad. Sci. USA, 90:7889-7893 [1993]).

The formulation herein may also contain more than one active compound as necessary for the particular indication being treated, preferably those with complementary activities that do not adversely affect each other. Such molecules are suitably present in combination in amounts that are effective for the purpose intended.

The active compounds may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (e.g., poly(2-hydroxyethyl-methacrylate), or poly(vinylalcohol)), polylactides (U.S. Pat. No. 3,773,919), copolymers of L-glutamic acid and ‘y-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

U.S. Pat. No. 6,692,738 describes delivery systems comprising implantable matrices seeded with cells genetically engineered to express MIS. Such implants were shown to produce bioactive MIS in supraphysiologic concentrations, reducing ovarian tumor growth in mice. Similar in-vivo delivery systems are applicable to the present invention.

A number of gene therapy methods relating to the administration of nucleic acid sequences encoding MIS have been described in U.S. Pat. No. 6,673,352.

For example, cells from a patient may be engineered ex vivo with a polynucleotide (DNA or RNA) comprising a promoter operably linked to an MIS polynucleotide as set forth in SEQ ID NO:1 or SEQ ID NO:3 or C-terminal fragments thereof, with the engineered cells then being provided to a patient to be treated with the polypeptide. Alternatively, the gene constructs bearing the MIS polynucleotide may be directly administered to the cells of an animal by any method that delivers injectable materials.

In preferred embodiments the modes of administration may involve the use of gene constructs operatively linked to a neuron- or glia-specific promoter such as neuron-specific enolase (neuron-specific) or glial fibrillary acidic protein (GFAP) (Harrop J S et al., Spine, [2004] 29(24):2787-92; Navarro V et al., Gene Therapy, [1999] 6(11):1884-92.)

The precise nature of the carrier or other material will be dependent upon the desired nature of the pharmaceutical composition, and the route of administration e.g. oral, intravenous, cutaneous, subcutaneous, intradermal, intramuscular, intra-articular, intrasynovial intraperitoneal or intracerobrospinal.

In immunoadjuvant therapy, the pharmaceutical composition will be in the form of a parenterally acceptable aqueous solution which is pyrogen-free and has suitable pH, isotonicity and stability.

When treating motor neuron diseases, brain specific modes of administration may be used, such as direct injection into the brain, including intrathecal or intraventricular injection. Alternatively, gene constructs may obtain entry to the brain by retrograde axonal transport after injection into the periphery, for example following intramuscular or intravenous injection.

It is also possible that MIS, the C-terminal fragment thereof, or a MIS receptor may be administered to the brain via the blood-brain barrier. Despite the classical perception that protein factors cannot pass this barrier, other TGFβ molecules which share a number or characteristics of molecular structures with MIS have been known to cross through the blood-brain barrier.

Drug delivery across the blood-barrier has been a focal point of pharmnaceutical science that is directly linked to the development of new therapeutic treatment. A number of methods have been tested at clinical trial levels (reviewed e.g., by Kemper et al., [2004] Cancer Treatment Rev., 30:415-423).

Administration of the pharmaceutical composition of the invention is preferably in a “therapeutically-effective amount”, this being sufficient to show the desired benefit to the individual. The actual amount administered, and rate and time-course of administration, will depend on the nature and severity of the underlying condition. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Oslo, A. (ed), 1980.

The effective amount may vary depending upon criteria such as the age, weight, physical condition, past medical history, and sensitivity of the recipient. The effective amount will also vary depending on the route of administration, for example oral, intravenous, intramuscular, subcutaneous, local, or by direct application to the brain.

In methods of enhancing neuronal cell survival in vitro, the method comprises culturing the neuronal cells in the presence of at least one MIS receptor agonist or antagonist. Motor neuron cultures can be prepared following protocols well known in the art and as described herein in the Examples. Immediately following seeding, MIS can be added to promote neuron growth and differentiation using standard cell culture techniques.

In preferred embodiments the MIS receptor agonist or antagonist is Müllerian inhibitory substance (MIS) or a C-terminal fragment thereof, functional derivatives or analogs thereof, or an antibody or antibody fragment that binds thereto.

In methods of the invention, it is preferable to achieve a serum or medium concentration of at least 1 ng/ml of MIS or a C-terminal fragment thereof. Preferably the concentration ranges from about 1 ng/ml to about 20 μg/ml of MIS or a C-terminal fragment thereof, preferably from about 10 ng/ml to about 500 ng/ml, preferably from about 50 to about 100 ng/ml. It is expected that the agonists or antagonists of the invention may be therapeutic across a range of doses, with a preference towards dose regimes that reproduce the levels of MIS in male fetuses and boys.

The concentration of MIS in the serum of boys before puberty is 40-90 ng/ml. It drops to a low level 3.7-6.9 ng/ml in adults (Lee M M et al., J. Clin. Endocrinol. Metab., 81:571-576.)

A number of studies describe the use of therapeutic doses of MIS in the treatment of varying disease states.

For the direct application to a tumor, U.S. Pat. No. 6,673,352 (specifically incorporated herein by reference in its entirety) describes a serum concentration of MIS protein or the C-terminal fragment thereof ranging from 1 ng/ml to about 20 μg/ml. In the treatment of excess androgen states, a single injection of 1 mg MIS was found to lower testosterone concentration in rats.

U.S. Pat. No. 5,198,420 describes a subcutaneous injection of 1 μg MIS for the treatment of neonatal respiratory distress syndrome, equating to a serum concentration of approximately 1 μg/ml.

U.S. Pat. No. 6,692,738 describes implants of varying sizes, each comprising a matrix seeded with cells genetically engineered to express MIS and implanted into the ovarian pedicle of mice. 14 days after implantation, the serum levels of MIS measured 100-500 ng/ml and at 28 days the levels measured 7-10 μg/ml. Implants secreting MIS were shown to significantly reduce ovarian tumor growth in mice compared with the implant alone.

Gupta et al. (2005) describes the use of MIS to suppress tumour growth in the C3T(1)T antigen transgenic mouse mammary carcinoma model. In these experiments, 20 μg MIS was administered per animal for five days a week with a treatment-free interval of 2 days over a six week period. MIS was shown to inhibit the growth of spontaneously arising mammary tumours.

The invention will now be described in greater detail by reference to specific Examples, which should not be construed as in any way limiting the scope of the invention.

### INDUSTRIAL APPLICATION

The methods, uses and pharmaceutical compositions of the present invention are particularly useful in modulating motor neuron function and activity, through the administration of at least one of the disclosed MIS receptor agonists or antagonists. Such embodiments are applicable particularly but by no means exclusively to treat motor neuron disease.

All of the references mentioned above are hereby incorporated by reference in their entirety.

Although the invention has been described by way of example and with reference to particular embodiments, those persons skilled in the art will understand that the invention is not limited thereto. It is to be understood that modifications and/or improvements may be made without departing from the scope or spirit of the invention.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Animals

All experiments were approved by the University of Otago's Animal Ethics Committee. Adult C57B 16 mice (5-8 week) and 14-day pregnant dams were bred at the University of Otago.

Laser Capture Microdissection (LCM)

Lumbar spinal cords were embedded in OCT embedding medium (Tissue-Tek®, VWR, San-Diego, Calif., USA), snap frozen in liquid-nitrogen cooled isopentane and stored at −80° C. until sectioned at a thickness of 10 μm in a cryostat. The sections were stained with 0.5% cresyl violet acetate for two minutes and dehydrated in ethanol (75% ethanol for 15 seconds, 95% ethanol for 15 seconds and 100% ethanol for one minute) and transferred to xylene for two minutes. The PixCell 2 LCM System and CapSure HS LCM Cap (Arcturus Engineering, Mountain View, Calif., USA) were used to dissect motor neurons. The laser power was set at 75 mw with a 7.5 μm aperture and 0.8 ms laser fire duration. The cell bodies of the motor neurons were identified based on their ventral-lateral location, large nuclear diameter (>9-10 μm) and intense staining with cresyl violet. Typically 400-500 motor neurons were collected on 3 to 4 caps, from each mouse.

The absence of glia in samples was confirmed.

RNA Preparation, cDNA Synthesis and Real-Time PCR

Total RNA fractions were isolated using TRIzol reagent (Invitrogen, Carlsbad, Calif., USA) for adult mouse tissues or PicoPure™ RNA isolated kit (Arcturus Engineering) for LCM samples following the manufacturer's protocols. The isolated RNA fractions were initially treated with DNase I (Promega, Madison, Wis., USA) to remove genomic DNA contamination. The cDNA was synthesized with SuperScript II RNaseH⁻ (Invitrogen) and oligo-d(T)₁₅ as the primer. The real-time PCR reactions were performed using an ABI Prism 7000 (Applied Biosystems), SYBR Green Master Mix (Applied Biosystems) and gene specific primers (Table 1).

TABLE 1

OLIGONUCLEOTIDE PRIMER SEQUENCES

| | | | |
|---|---|---|---|
| | | Accession | Size |
| Primer | Sequence | # | (bp) |

| | | | |
|---|---|---|---|
| ALK1-F | 5′-ACAAAGCAGCGATTACCTGGATA | NM_009612 | 190 |
| | G-3′ (SEQ ID NO:5) | | |
| ALK1-R | 5′-GTAATCCTCCACAATGCCATTGA | | |
| | T-3′ (SEQ ID NO:6) | | |
| ALK2-F | 5′-GATCAACAGAGGCCAAACATACCT | NM_007394 | 274 |
| | A-3′ (SEQ ID NO:7) | | |
| ALK2-R | 5′-AGATGGATTCTGTTCTGACAACC | | |
| | A-3′ (SEQ ID NO:8) | | |
| ALK3-F | 5′-GTCTATTCCAGGGCAGATTTCCT | BC042611 | 160 |
| | A-3′ (SEQ ID NO:9) | | |
| ALK3-R | 5′-CCTGCTTAACATCTGACGCAAG | | |
| | T-3′ (SEQ ID NO:10) | | |
| ALK4-F | 5′-TTGAGGAGATGCGAAAGGTTGTA | BC059832 | 179 |
| | T-3′ (SEQ ID NO:11) | | |
| ALK4-R | 5′-CACGCTTAGCTGGGACAGAGTC | | |
| | T-3′ (SEQ ID NO:12) | | |
| ALK5-F | 5′-ATCTATGCAATGGGCTTAGTGTTC | NM_009370 | 154 |
| | T-3′ (SEQ ID NO:13) | | |
| ALK5-R | 5′-GCCTTAACTTCTGTTCGCAAACTA | | |
| | CT-3′ (SEQ ID NO:14) | | |
| ALK6-F | 5′-ATACCAGCTTCCCTATCACGACC | NM_007560 | 277 |
| | T-3′ (SEQ ID NO:15) | | |
| ALK6-R | 5′-TGAAATTCTTGCTCTGTCCACAAG | | |
| | TA-3′ (SEQ ID NO:16) | | |
| ALK7-F | 5′-CCATTGCTTTGCTCCTTATGGAT | XM_194020 | 174 |
| | A-3′ (SEQ ID NO:17) | | |
| ALK7-R | 5′-GGCAACAAATTCATGCTCAAATGT | | |
| | A-3′ (SEQ ID NO:18) | | |
| GAPDH-F | 5′-CTTCATTGACCTCAACTA-3′ | NM_008084 | 300 |
| | (SEQ ID NO:19) | | |
| GAPDH-R | 5′-TTCACACCCATCACAAAC-3′ | | |
| | (SEQ ID NO:20) | | |
| GFAP-F | 5′-GTGCTCCTGGTTCGAGTCCTTA | NM_010277 | 162 |
| | A-3′ (SEQ ID NO:21) | | |
| GFAP-R | 5′-AAGCGCATCACGATTCCTGTAC | | |
| | A-3′ (SEQ ID NO:22) | | |
| MIS-F | 5′-TGCGGGAGCTGAGTGTAGATC | NM_007445 | 118 |
| | T-3′ (SEQ ID NO:23) | | |
| MIS-R | 5′-TAGCGCGGATTACGGTCAGAC | | |
| | T-3′ (SEQ ID NO:24) | | |
| MISRII-F | 5′-CAGAGTTCTTGTCTCCTCAGTGT | NM_144547 | 158 |
| | T-3′ (SEQ ID NO:25) | | |
| MISRII-R | 5′-AGAGAATGAGAGCAGTAGGCATG | | |
| | T-3′ (SEQ ID NO:26) | | |
| RET-F | 5′-GCTTGTACATCGGGACTTAG-3′ | BC059012 | 158 |
| | (SEQ ID NO:27) | | |
| RET-R | 5′-ACTCAATTGGCATGCACTTGA-3′ | | |
| | (SEQ ID NO:28) | | |
| TβRII-F | 5′-CTTGGATGAGAGCAGGGAATG | BC052629 | 233 |
| | A-3′ (SEQ ID NO:29) | | |
| TβRII-R | 5′-GTGTTCGAAGCCACAGAACAC | | |
| | A-3′ (SEQ ID NO:30) | | |

The forward and reverse primers used for RT-PCR detection of the listed genes are indicated by “-F” and “-R”, respectively.

A two-step PCR reaction was carried out with denaturation at 95° C. for 15 seconds, annealing and extension combined at 60° C. for one minute in a total of 40-50 cycles. The uniqueness of amplicons was analyzed using dissociation curves and by sequencing (Centre for Gene Research, University of Otago). The copy numbers of each gene was calculated from a standard curve. The DNA used for each standard was amplified from a mouse spinal cord cDNA pool using PCR reactions and purified using gel electrophoresis and QlAquick Gel Extraction Kit (Qiagen, Valencia, Calif., USA) following the manufacturer's protocol.

Microarray

A full description of the methods for the gene expression profiling with microarray are described in a thesis by Mike Kimber “The temporal pattern of motor neuron specific differential gene expression in an animal model of amyotrophic lateral sclerosis” University of Otago, 2003. A summary of the array methods is described below.

Motor neurons were isolated from the lumbar spinal cords of adult wild-type mice and adult SOD I mice, by laser microdissection, as described for the real-time PCR study.

Total RNAs were obtained from the isolated motor neurons using a PicoPure RNA Isolation Kit (Arcturus Engineering, Mountain View, Calif., USA) and treated with RQI RNase-free DNase (Promega, Madison, Wis., USA). cDNAs were prepared from the DNase-treated RNA samples, using a protocol described by Matz et al., (Nucl. Acids Res., 27:1558-1560, 1999) with 2 primers:

cDNA synthesis primer: 5′-AAGCAGTGGTATCAACGCAGAGT₃₀VN-3′ (SEQ ID NO:31); and

Template switch 5′-AAGCAGTGGTATCAACGCAGAGTACGC-3′), (SEQ ID NO:32) and SuperScript II RNase H- Reverse Transcriptase (Invitrogen Corporation, Carlsbad, Calif., USA).

The cDNAs were amplified by PCR using Advantage® 2 PCR Kit (Clontech, Paroalt, Calif., USA) with a LD-PCR primer (5′-AAGCAGTGGTATCAACGCAGAGT-3′) (SEQ ID NO:33). The amplification cycles consisted of 15 seconds at 95° C., 15 seconds at 65° C. and 6 minutes at 68° C. The optimal number of cycles was determined by checking aliquots of the reaction mixtures by agarose-electrophoresis.

The cDNAs were converted into aminoallyl-dUTP (aa-dUTP)-labelled cDNA fragments using a BioPrime® DNA labelling kit (Invitrogen Corporation, Carlsbad, Calif., USA) which was based on the random prime labelling method (Feinberg, A. P. and Vogelstein, B. (1983). “A technique for radiolabeling DNA restriction endonuclease fragments to high specific activity.” Anal Biochem 132:6-13).

The aa-dUTP-labelled cDNA fragments prepared from the wild-type and SOD motor neurons were differentially coupled with either monofunctional Cy3 or Cy5 dyes (Amersham Biosciences), and hybridized together on the National Institute for Aging 15k mouse microarray slides (Australian Genome Research Facility, Melbourne, Australia) and another slides containing a 5K murine and a 1.8K rat set of verified cDNAs (Otago Genomics Facility, University of Otago, Dunedin, New Zealand). After hybridisation and washing, the arrays were scanned with a ScanArray 5000 Microarray Analysis System (Packard BioChip Technologies, Billerica, Mass., USA). The resulting images were then processed with Genepix Pro 4.1 (Axon Instruments, CA) and transferred to Gene Traffic (Iobion Informatices, la Jolla, Calif., USA). The data was normalised by the Lowess method (Cleveland, W. S. [1979] J. Amer. Statistical Assoc., 74:829-836). The array data was then scanned to discover genes that were: (1) known to be growth factor receptors; (2) not known to be expressed by neurons; (3) abundant in motor neurons.

Samples of six male and five females were examined using real-time PCR, without pre-amplification of the mRNA and using standards so that the copy number could be calculated. The copy numbers of various genes for each mouse were compared after normalisation to the copy number of GAPDH.

MISRII was present in both male and female motor neurons with a copy number greater than the receptors for the classical motor neuron survival factors (e.g., RET, TPRII).

ALK2, 3 and 6 were also present, but with a lower copy number than for MISRII. In the inventors' experience, it is usual for the type I TGF-β-superfamily receptor to have a lower abundance than for the corresponding type II receptor.

Immunoprecipitation and Western Blotting

Murine whole spinal cords and testes were crushed and lysed in buffer containing 150 mM NaCl, 20 mM Tris-HCl, pH 7.4, 1% sodium deoxycholate, 1% SDS, 1% NP-40 and 10% Complete™ solution (Boehringer Mannheim, Mannheim, Germany). Immunoprecipitation was carried out with MISRII antibody (2.5 μg/ml, R&D System) overnight at 4° C. followed by adsorption of protein G-Sepharose (Sigma) for 2 h at 4° C. Immunoprecipitates were washed three times in wash buffer (150 mM NaCl, 20 mM Tris-HCl, pH 7.4) and eluted by boiling in LDS NuPAGE® LDS sample buffer (Invitrogen) and NuPAGE® LDS reducing agent (Invitrogen) for 5 min. Proteins were separated by electrophoresis of NuPAGE® Bis-Tris gels (Invitrogen) and transferred onto 0.2 μm polyvinylidene fluoride membranes (Invitrogen). Blocking buffer containing 1% casein (Amersham) and 0.04% Tween 20, MISRII antibody (1:500, R&D System) and peroxidase-conjugated anti-goat IgG antibody (1:10,000 Jackson ImmunoResearch, West Grove, Pa., USA) were used for Western blotting. Proteins were visualized by chemiluminescence (ECL Plus kit, Amersham).

A band with an appropriate MW of 65kDa was detected in the spinal cord and testes. This is consistent with the presence of MISRII protein.

Immunohistochemistry

Transverse sections of lumbar spinal cords and selected tissues were cut in a cryostat at a thickness of 10 μm. The sections were stained by immunohistochemistry as previously described (Russell et al., 2000). Briefly, the sections were fixed in 1% neutral buffered paraformaldehyde at 4° C. and incubated with MISRII (1:50, R&D System), MIS (1:25, R&D System) and ALK3 (1:15, R&D System) specific primary antibody overnight at 4° C. The slides then sequentially incubated with a biotinylated anti-goat IgG antibody (1:75, Sigma, St Louis, Mo., USA), methanol/H₂O₂, and finally a streptavidin biotinylated-horseradish peroxidase complex (1:200, Amersham). The immunoreactivity was visualized using 3-amino-9-ethylcarbamide as the chromogen (Sigma). In all experiments, the primary antibody was replaced by a non-immune goat IgG (Sigma) as a control for non-specific binding.

Strong MISRII-immunoreactivity was detected in motor neurons (perinuclear stain, which is assumed to be Golgi). Glia were negative but some non-motor neurons were also positive. ALK3-immunoreactivity was also detected in motor neurons, but the intensity of this stain was less than for MISRII.

Results

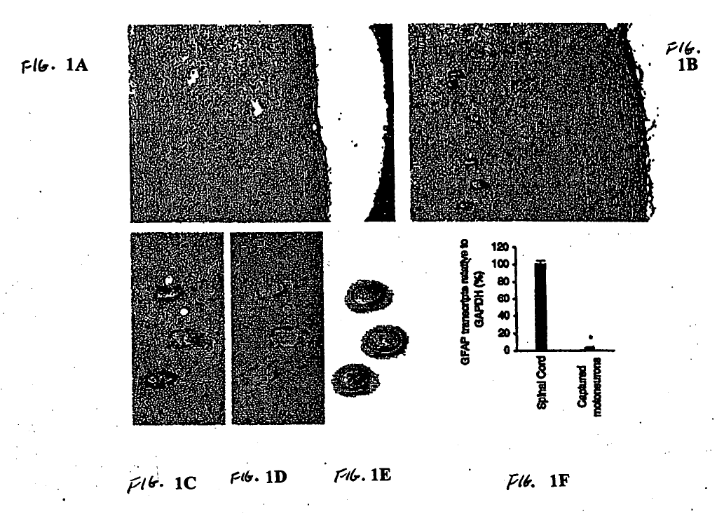

The cell bodies of 500-600 spinal motor neurons per mouse were isolated from five female and 6 male mice, using laser capture microdissection (FIG. 1A through FIG. 1E). Motor neuron cell bodies have an unusually high abundance of mRNA. Consequently, sufficient mRNA was collected from each mouse for multiple genes to be analysed by real time PCR, without any pre-amplification of the mRNA. All of the samples had minimal contamination from neighbouring glia, as evidenced by the low abundance of an astrocyte-specific transcript (FIG. 1F).

The mRNA of the type II receptor for MIS (MISRII) was plentiful in the isolated motor neurons. The copy number of MISRII transcripts was measured and normalised relative to that of GAPDH transcripts. The abundance of MISRII in isolated motor neurons and the testes were an order of magnitude greater than for other tissues (FIG. 2). In particular, the levels of MISRII transcripts in the isolated motor neurons were 30 times higher than in the whole spinal cords and brain, indicating that most neurons and glia have minimal levels of MISRII. This may explain why MISRII has not previously been detected in the nervous system. The levels of MISRII transcripts were also low in other parts of the neuromuscular system (sciatic nerve and skeletal muscle).

MISRII can exist in a short form, which lacks the MIS binding site (di Clemente et al., 1994). RT-PCR analysis shows the MISRII in motor neurons were predominantly the full-length transcripts, with only trace levels of the short form being detected.

MIS, like other members of the TGF-P superfamily, signals through a complex consisting of type I and II receptors (Caestecker, M. D. [2004] Cytokine Growth Factor Rev, 15:1-11. Josso, N. and N. Clemente [2003] Trends Endocrinol. Metab., 14:91-97.

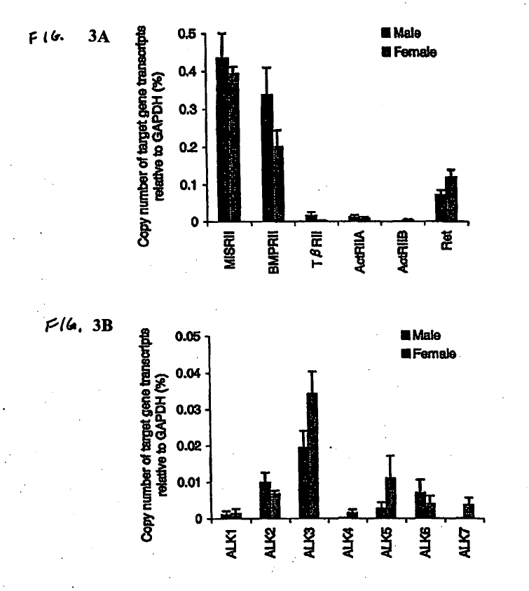

There are seven type I receptors in mammals for the TGF-β superfamily, ALK1-7. The most abundant of the seven ALK receptors in isolated motor neurons was ALK3 (FIG. 3B), which is known to mediate the developmental actions of MIS in mice (Josso, N. and N. Clemente [2003]).

The copy number of ALK3 in motor neurons was, however, significantly less than for the copy number of MISRII (FIG. 3A). ALK2 and ALK6 are putative receptors for MIS (Josso, N. and N. Clemente (2003) and were present in motor neurons at lower levels (FIG. 3B), raising the possibility of MIS being able to mediate diverse actions on motor neurons (see for example, Josso, N. and N. Clemente [2003]).

The copy number of MISRII in motor neurons was then compared to the abundance of the receptors for TGF-β2 and GDNF, which are known potent regulators of motor neuron survival (Jiang et al., [2000] Neuroscience, 97:735-742). The abundance of MISRII was 40 times that of the TPRII (receptor for TGF-β1-3), and 5 times that of RET (receptor for GDNF) (Sariola, H. and M. Saarna [2003] J. Cell Sci., 116:3855-62.). Similarly, the abundance of the MIS type I receptors, ALK3 was far more abundant than the type I TGF-β subfamily receptors, ALK5 and ALK6.

MIS has male-specific functions during the sexual differentiation of embryos and the frequencies of motor neuron disorders have small unexplained gender differences. The abundances of receptors in male and female motor neurons were therefore compared. All of the receptors were present in both sexes, with slight variation detected in the abundance of ALK3 (p=0.05) and RET (p=0.04).

MISRII and ALK3 Proteins are Produced by Motor Neurons

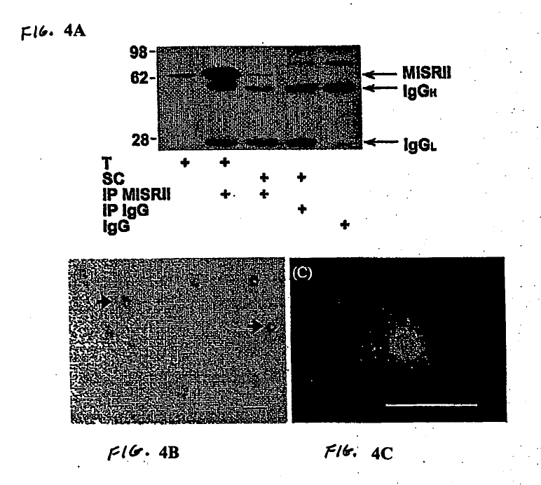

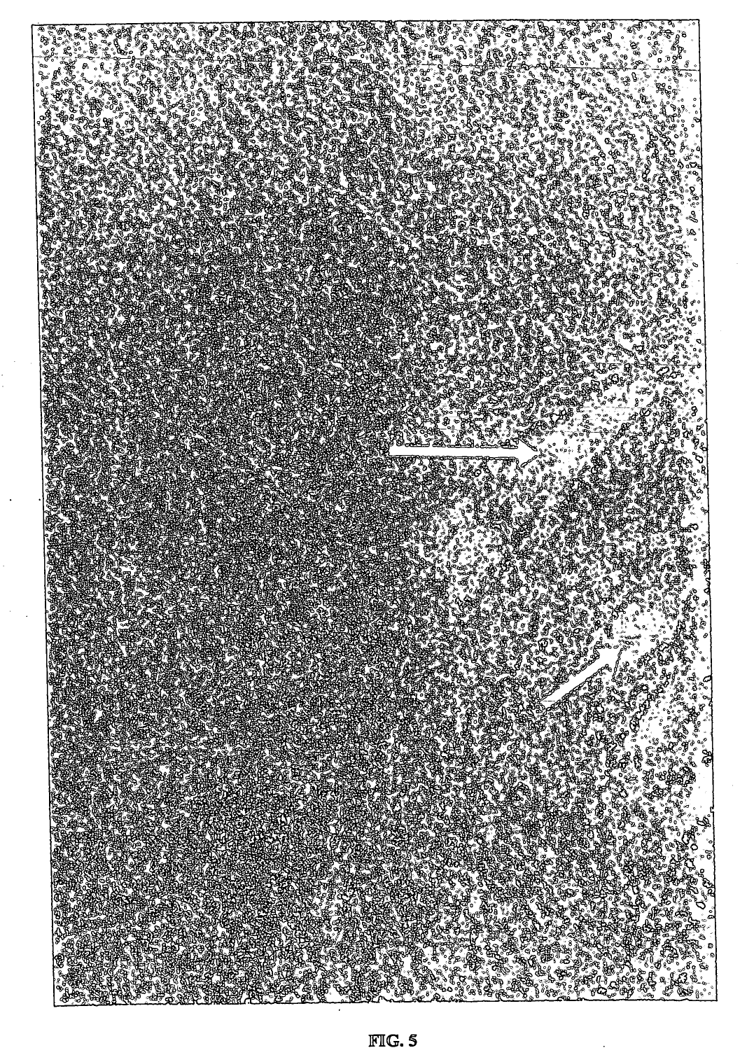

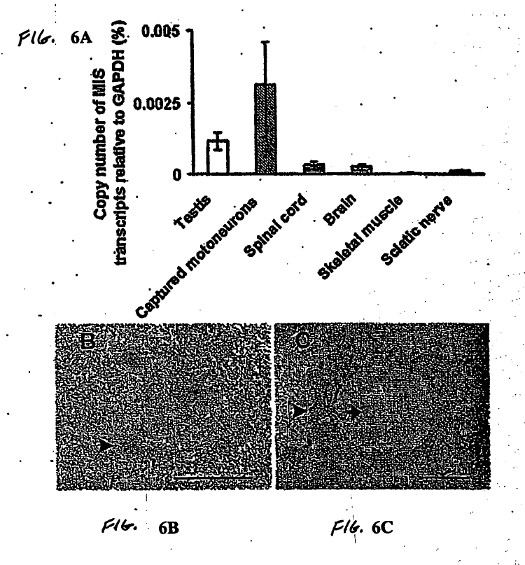

The inventors then examined whether proteins are produced from the MIS receptor mRNAs. A 63-kDa protein was immunoprecipitated with an anti- MISRII antibody from both the spinal cord and testes (FIG. 4A). This molecular weight is consistent with previous studies of the MISRII. The anti-MISRII antibody was then used to localise the MISRII protein.

Strong immunoreactivity was detected in spinal lumber motor neurons (FIG. 4B), with lesser levels being associated with some other spinal neurons and a minority of neurons in the brain. The MISRII-immunoreactivity was perinuclear and appeared to be associated with the endoplasmic reticulum/Golgi apparatus of the neurons; such staining is commonly seen with antibodies to growth factors and their receptors. Much lower levels of MISRII-immunoreactivity were associated with the circumferences of the neurons (arrowheads), which is consistent with staining of the plasma membrane (FIG. 4C).

Motor neurons were also stained by an antibody to ALK3. In apparent reflection of the mRNA abundance, the intensity of the ALK3 immunoreactivity was noticeably less than that of the MISRII immunoreactivity. The ALK3 immunoreactivity was associated with the plasma membrane of the motor neuron (FIG. 5).

MIS Transcripts and Proteins are Present in Motor Neurons

If the ALK3/MISRII receptors have a physiological function in motor neurons then a source of MIS should be present in the neuromuscular system. Only trace levels of MIS mRNA were detected in skeletal muscle, the sciatic nerve, the spinal cord and the brain, using real time PCR (FIG. 6A). The levels of MIS transcripts in the isolated motor neurons were, in contrast, comparable to the abundance of MIS in the testes (FIG. 6A). No sex difference was observed in the level of MIS in motor neurons. This was then further verified using immunochemistry. The anti-MIS antibody stained motor neurons (FIG. 6B) and a minority of other neurons, as well as the classical sources of the MIS (Bezard et al., 1987; Tran et al., 1987), the Sertoli cells of the testes and ovarian granulosa cells (FIG. 6C) as described previously.

Mouse Motor Neuron Culture

The spinal cords of 14-day-old (E14) embryos were dissected and incubated for 15 minutes at 37° C. in Dulbecco's phosphate buffered saline (DPBS, pH=7.2, Sigma) containing 10 μM beta-mercaptoethanol (Sigma), 0.05% trypsin (Sigma) and 0.04% EDTA (Sigma). The digestion was then stopped by the addition of 0.033% trypsin inhibitor (Sigma) and the cords were mechanically dissociated by drawing them up-and-down several times through a 21- and then a 23-gauge needle.

The resulting cell suspension was passed through a mesh (pore size=100 μm, Sigma), overlaid onto 10.4% Optiprep (Sigma) in DPBS and centrifuged for 20 minutes at 400×g at room temperature. The upper phase, containing the purified motor neuron was collected and centrifuge for 10 minutes at 700×g at room temperature. The purified motor neurons (2000 cells/cm²) were cultured under serum-free conditions in Neurobasal medium (Invitrogen) supplement with B27 and 500 μM glutamine at 37° C. in a humidified atmosphere with 5% CO₂.

Recombinant MIS and hGDNF (Alomone Labs, Israel) were added immediately after seeding. Twenty-four hours later, the numbers of surviving motor neurons with axons were counted in three randomly selected fields using a phase-contrast microscope.

Half the volume of medium was changed after two days. Four days after plating, the cultures were stained with antibody to the motor neuron marker, anti-Islet-1 (39.4D5, Developmental Studies Hybridoma Bank) and the immunoreactivity developed as above, using biotinylated-anti-mouse IgG (Jackson ImmunoResearch Laboratories, USA). The numbers of surviving motor neurons were determined by counting Islet-1^{+ve} neurons with a neurite in three randomly selected fields in each well. Three wells were used for each concentration of factor and experiment was replicated four times. Virtually all cells were stained by Islet-I indicating the cultures were pure.

Purification of rhMIS

Bioreactive rhMIS was immunoaffinity-purified from CHO cells as described earlier (Ragin, R. C. et al., [1992] Protein Expression Purif., 3:236-45.) and its potency validated in a MIS-specific Müllerian duct regression assay (MacLaughlin, D. T. et al., [1991] Methods Enzymol., 198:358-69.). The MIS produced by this method is 140 kDa (70 kDa disulfide linked homodimer) that has been activated by proteolytic processing prior to secretion. The 25-kDa carboxy-terminal fragment, in which bioactivity resides, remains in non-covalent association with the amino terminus.

In Vitro Results

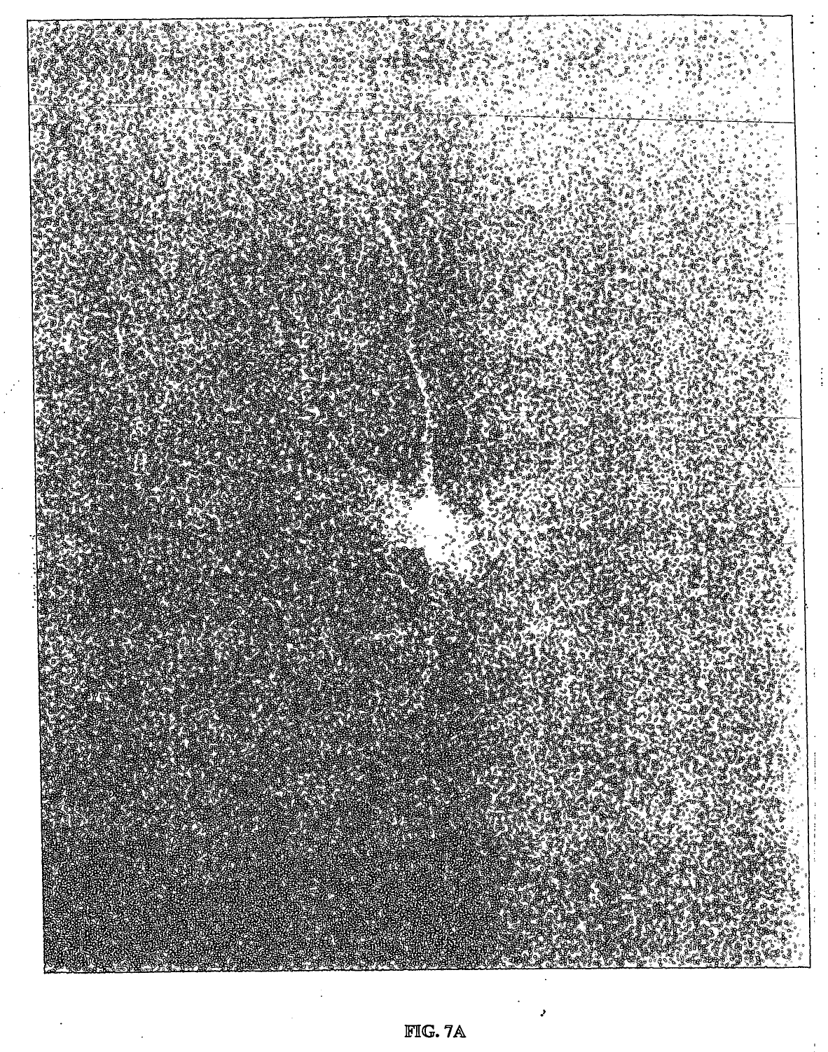

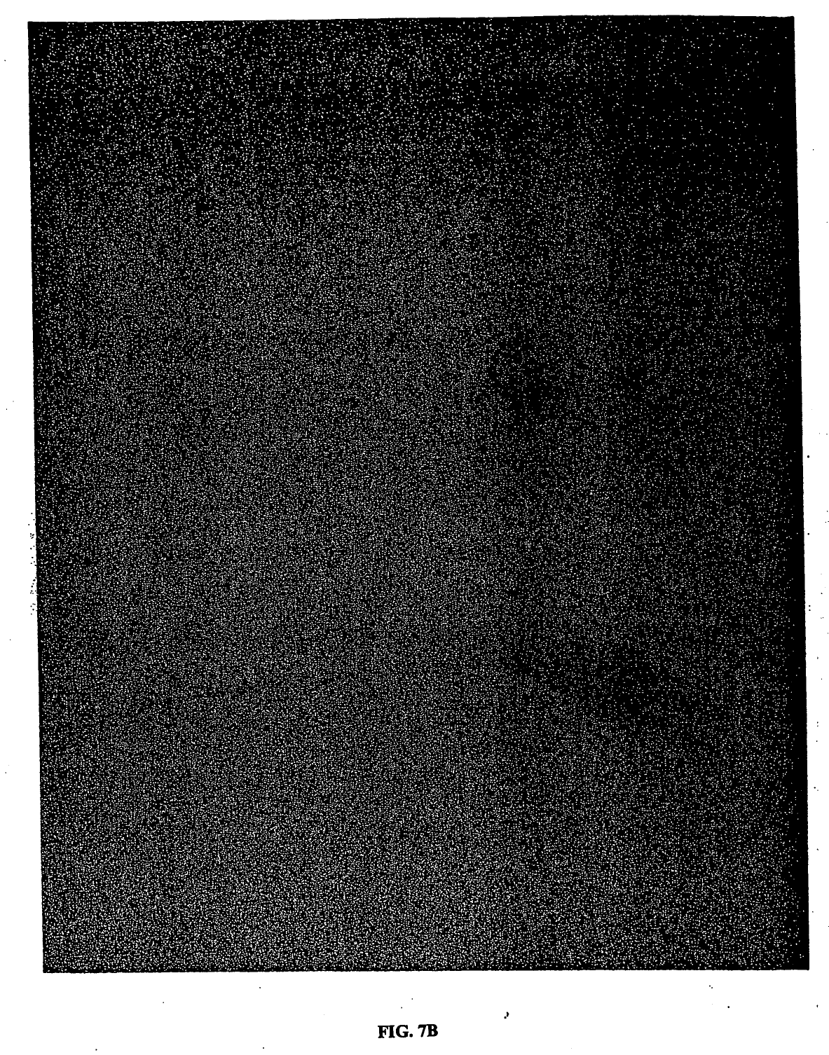

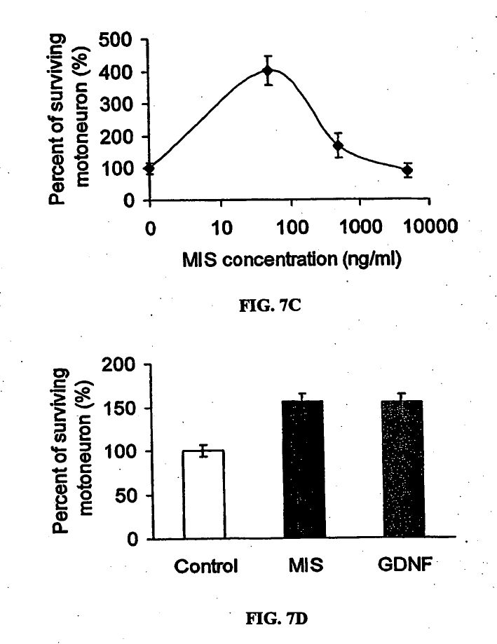

In the cultures with no added growth factor, approximately half of the neurons had died and floated into the medium, by 24 hours. Most of the remaining neurons lacked neurites, and may have also been dead. A few of these neurons had extended a simple neurite that lacked branches (FIG. 7A).

After MIS treatment, there was a dose dependent increase in the number of neurons. Most of the neurons had multiple neurites, which were usually highly branched (FIG. 7B). Neurons with this morphology were not seen in control cultures.

The effect of MIS was biphasic, with the maximum effect being at a concentration of 50-100 ng/ml. The MIS-induced survival persisted for up to 2 weeks, the longest time tested.

The number of neurons with neurites is illustrated in FIG. 7C. The inhibitory effect of very high non-physiological doses of MIS occurs with other members of the TGF-β superfamily. The maximal effect of MIS was greater than that produced by 10 μg/ml of GDNF, a known motor neuron survival factor.

Historic assessments of putative neuronal survival factors have variably counted differentiated neurons and total neuron number. The later method gives lower signal to noise ratios, as it includes healthy and dying neurons. Nevertheless the conclusions reached with the two methods are usually comparable. Total cell counts are illustrated from a replicate experiment in FIG. 7D. The total number of surviving neurons was similar in the cultures treated with 50 ng/ml of MIS or 50 ng/ml of GDNF.

The concentration of MIS in the serum of boys before puberty is 40-90 ng/ml. It drops to a low level 3.7˜6.9 ng/ml in adults (Lee M M et al., J. Clin. Endocrinol. Metab., 81:571-576.) The effective concentration of MIS in the motor neuron cultures was therefore in the physiological range.

The results described in the present invention strongly suggest that MIS is a regulator of mature motor neurons. Motor neurons produce MISRII, which is the unique receptor for MIS, as well as the three type I receptors that associate with MISRII. Furthermore, MIS supported the survival and differentiation of embryonic motoneurons in vitro, indicating that activation of MIS receptors leads to downstream functional consequences in motor neurons. In the TGF-β superfamily, the type II receptor determines ligand and specificity, whereas the type I receptors controls downstream pathway activation.

Human and murine MIS^{-/-} or MISRII^{-/-} null mutants do not exhibit overt motor symptoms. There are two possible reasons for this. First, the differentiation and maintenance of motor neurons appears to be controlled by multiple redundant factors (Oppenheim, R. W., [1996] Neuron, 17:195-197.). The absence of any one of these factors only produces a partial loss of motor neurons, which is subclinical (e.g., GDNF^{-/-} (Oppenheim, R. W., et al., [2000] J. Neurosci., 20:5001-11.)). This contrasts with DRG or sympathetic neurons where the null mutation of their neuronal survival factors produces massive cell death (Crowley, C., et al. [1994] Cell, 76:1001-1011.). Second, some neuronal survival factors appear to have no role in the day-to-day function of the neuron, but are vital when the neuron is subject to an insult. For instance, interleukin-6 (IL6) is a potent neuronal survival factor, but the brains of IL6^{-/-} mice are normal (Galiano, M., et al., [2001] Eur. J Neurosci., 14:327-41) until a virus penetrates the blood brain barrier (Pavelko, K. D., et al., [2003] J Neurosci., 23:481-92.). Similarly, the importance of VEGF to motor neurons appears to be limited to transient episodes of hypoxia: consequently, VEGF deficiency only leads to motoneuron loss and motor disease once the nervous system has aged (Oosthuyse, B. et al., [2001] Nature Genet., 28:131-8.).

Without wishing to be bound by theory it is considered that MIS affects neurons by activation of the type II Müllerian inhibitory substance receptor (MISRII) and a co-receptor. The presence of MIS receptors in motor neurons indicates MIS may be important in various neurodegenerative conditions and/or in psychiatric disorders.

The location of MIS and demonstrated effect on survival and differentiation of embryonic motor neurons is consistent with MIS being an autocrine regulator of motor neurons and/or a mediator of motor neuron-to-motor neuron communication.

Since motor neuron-to-motor neuron interactions are known to be more prevalent after damage to motor axons/nerves (Chang, Q. et al., [1996] Genes Dev., 10:2577-87), it is believed that MIS may be important in the survival and functioning of neurons after injury. Therefore, MIS has potential as a therapy for motor neuron disorders.

MIS may also play a number of roles as an agent for treating neuro-degenerative disorders such as Amyotrophic lateral sclerosis, progressive muscular atrophy, postpolio syndrome and sarcopenia.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of modulating motor neuron activity in a patient in need thereof, said method comprising at least the step of administering to said patient a therapeutically-effective amount of at least one Müllerian inhibitory substance receptor agonist or antagonist for a time sufficient to modulate said motor neuron activity in said patient.

2. The method of claim 1, wherein modulating said motor neuron activity enhances neuronal cell survival in said patient.

3. The method of claim 2, wherein said patient has suffered an injury to the brain.

4. The method of claim 1, wherein said patient has a condition or disease characterized by motor neuron cell death or impairment.

5. The method of claim 4, wherein said condition or said disease is a motor neuron disease selected from the group consisting of amyotrophic lateral sclerosis (ALS), progressive muscular atrophy, postpolio syndrome and sarcopenia.

6. The method of claim 1, wherein said Müllerian inhibitory substance receptor is a Müllerian inhibitory substance type II receptor, a Müllerian inhibitory substance type I receptor, or a combination of both type I and type II receptors.

7. The method of claim 6, wherein said Müllerian inhibitory substance receptor is a Müllerian inhibitory substance type II receptor.

8. The method of claim 1, wherein said Müllerian inhibitory substance receptor agonist or antagonist is a Müllerian inhibitory substance polypeptide, peptide, variant, fragment or analog thereof; or an antibody or antibody fragment that specifically binds to said Müllerian inhibitory substance polypeptide.

9. The method of claim 1, wherein said Müllerian inhibitory substance receptor agonist or antagonist is formulated for delivery to a mammalian brain.

10. The method of claim 1, wherein said Müllerian inhibitory substance receptor agonist or antagonist is formulated for administration to a human.

11. A method of enhancing neuronal cell survival in-vitro, said method comprising at least the step of culturing a population of neuronal cells in the presence of at least one Müllerian inhibitory substance receptor agonist or antagonist in an amount and for a time sufficient to enhance said neuronal cell survival in vitro.

12. A pharmaceutical composition comprising at least one Müllerian inhibitory substance receptor agonist or antagonist and a pharmaceutically-acceptable carrier or excipient.

13. The pharmaceutical composition of claim 12, formulated for administration to a patient that has suffered an injury to the brain.

14. The pharmaceutical composition of claim 12, formulated for administration to a patient that has a condition or disease characterized by motor neuron cell death or impairment.

15. The pharmaceutical composition of claim 14, wherein said condition or said disease is motor neuron disease.

16. The pharmaceutical composition of claim 15, wherein said motor neuron disease is selected from the group consisting of amyotrophic lateral sclerosis (ALS), progressive muscular atrophy, postpolio syndrome and sarcopenia.

17. The pharmaceutical composition of claim 12, wherein said Müllerian inhibitory substance receptor is a Müllerian inhibitory substance type II receptor, a Müllerian inhibitory substance type I receptor, or a combination of type I and type II receptors.

18. The pharmaceutical composition of claim 17, wherein said Müllerian inhibitory substance receptor is a Müllerian inhibitory substance type II receptor.

19. The pharmaceutical composition of claim 17, wherein said Müllerian inhibitory substance receptor agonist or antagonist is Müllerian inhibitory substance or a biologically-active fragment thereof, or an antibody or an antigen binding fragment that specifically binds to said Müllerian inhibitory substance receptor agonist or antagonist.

20. The pharmaceutical composition of claim 19, wherein said Müllerian inhibitory substance receptor agonist or antagonist is formulated for administration to a mammalian brain.


SEQUENCE LISTING (4 sequences)

SEQ ID NO: 1 - DNA, 1906 bp, Bovine
  ref <308>: DATABASE ACCESSION NUMBER: Genbank/M13151
  ref <309>: DATABASE ENTRY DATE: 1996-05-01
  ref <313>: RELEVANT RESIDUES: (1)..(1906)
caaggtcatgtcccaggaggagatagggaccgccctgcaccacaaacagctctgctccctcttataaagtagggcagcccagcccctggaagctcccaggatgcccggtccatctctctctctggccctggtgctgtcggccatgggggctctgctgaggccagggacccccagggaagaagtcttcagcacctcagccttgcccagggagcaggccacaggcagcggggcactcatctttcagcaagcctgggactggccactctccagtctctggctgccaggcagccctctggaccccctgtgcctggtgaccctgcatgggagtggcaacgggagcagggcccccctgcgggtggtgggggtcctgagcagctacgagcaggccttcctggaggctgtgcggcgcacccactggggcctgagtgacttgaccaccttcgcagtgtgccccgctggcaacgggcagcctgtgctgccccacctgcagcggctgcaggcatggctgggggagcccggggggcggtggctggtggtcctgcacctggaggaagtgacgtgggagccaacacccttgctgaggttccaggagcctccgcctggaggagccagccccccagagctggcgctgctggtggtgtacccagggcctggcctggaggtcactgtcaccggggctgggctacctggcacccagagcctctgcctgaccgcggactcggacttcctggccttggtcgtggaccacccggagggggcctggcgccggcctgggttagcccttaccctgcggcgccgtggaaatggtgcgctcctgagcactgcccagctgcaggcgctgctgttcggtgcggactcccgctgcttcacacgaaagaccccagccctgttactcttgctgccggcccggtcttcggcaccgatgcccgcgcacggtcggctggacttggtgcccttcccgcagcccagggcttccccggagccagaggaggcaccgcccagcgctgatcccttcctggagactctcacgcgcctggtgcgcgcgcttgcgggacccccggcccgagcctcgccaccgcggctggccttggacccgggcgcactggctggtttcccgcagggccaggtcaacctgtcggaccccgcggccctggagcgcctgctggacggcgaggagccgctgctgctgctgctgccgccgacggcagccaccaccggggtccccgcaacgccgcaaggtcccaagtcccctctgtgggccgcgggactagcgcgccgggtggctgccgagcttcaggcggtggccgccgagctgcgtgccctcccggggctgcctccagctgccccaccgctgctggcgcgcctgctggcactgtgcccgggaaacccagacagccccggcggcccgctgcgcgcgctgctgctgctcaaagcgctgcagggcctgcgcgctgagtggcgcgggcgggagcggagcggctctgcacgggcgcagcgcagcgccggggccgcggctgcagacgggccgtgcgctctgcgtgagctgagcgtagacctgcgggccgagcgctcggtgctcatccccgagacataccaggccaacaactgccagggggcctgcggctggcctcagtcggaccgcaacccgcgctacggcaaccacgtggtgctgctgctaaagatgcaggcccgcggcgccaccctggcgcgcccgccctgctgtgtgcccacagcctacaccggcaagctcctcatcagcctgtccgaggagcgcatcagtgcgcaccacgtcccaaacatggtggccaccgaatgcggctgccggtgacctcgcgccgtgctcctcgtgctgccccggcccgtatttattcggaccccgtcattgccccattaaacacgggaaggc

SEQ ID NO: 2 - protein, 575 aa, Bovine
  ref <308>: DATABASE ACCESSION NUMBER: Genbank/M13151
  ref <309>: DATABASE ENTRY DATE: 1996-05-01
  ref <313>: RELEVANT RESIDUES: (1)..(575)
MPGPSLSLALVLSAMGALLRPGTPREEVFSTSALPREQATGSGALIFQQAWDWPLSSLWLPGSPLDPLCLVTLHGSGNGSRAPLRVVGVLSSYEQAFLEAVRRTHWGLSDLTTFAVCPAGNGQPVLPHLQRLQAWLGEPGGRWLVVLHLEEVTWEPTPLLRFQEPPPGGASPPELALLVVYPGPGLEVTVTGAGLPGTQSLCLTADSDFLALVVDHPEGAWRRPGLALTLRRRGNGALLSTAQLQALLFGADSRCFTRKTPALLLLLPARSSAPMPAHGRLDLVPFPQPRASPEPEEAPPSADPFLETLTRLVRALAGPPARASPPRLALDPGALAGFPQGQVNLSDPAALERLLDGEEPLLLLLPPTAATTGVPATPQGPKSPLWAAGLARRVAAELQAVAAELRALPGLPPAAPPLLARLLALCPGNPDSPGGPLRALLLLKALQGLRAEWRGRERSGSARAQRSAGAAAADGPCALRELSVDLRAERSVLIPETYQANNCQGACGWPQSDRNPRYGNHVVLLLKMQARGATLARPPCCVPTAYTGKLLISLSEERISAHHVPNMVATECGCR

SEQ ID NO: 3 - DNA, 2834 bp, Homo sapiens
  ref <308>: DATABASE ACCESSION NUMBER: Genbank/K03474
  ref <309>: DATABASE ENTRY DATE: 1996-05-03
  ref <313>: RELEVANT RESIDUES: (1)..(2834)
aaggtcgcggcagaggagataggggtctgtcctgcacaaacaccccaccttccactcggctcacttaaggcaggcagcccagcccctggcagcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcatcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaaggtatgtggggcccagccccaagcttggcaccgccgtcttccttcaggtgggccgggtcctcctagggaagatcaggggctggcagagcccccaccctgggcagggaggctgtggtcttgttcctaggactgggttgcgggtccgtggcctggaaggtgggcaccacactctgtcctgtccccgaagcccagctcttagacttgcccctgcctcggtgccagggagagagctgctgccttctccccacccctgaagacgacgcagggctcggggccagtggaacccttcttcccacagccccagcctgttctcagggccgctggcctaagatactccctgcggggaaggggcttcatcgggcaccccaacccagagaccccagggcggcagccccacccacagcctcagacgcagcccctgcctgcccctgccgtcaccgctccctggctgcaggaaggcagctaagaggggcacccttgtcccccgcttgaggtcccctgcacagtggccagagcggcagggacagatcccaaagattcccggggggtgtggccttcaatggctcaggcgtcccctgctgtcccggctgcagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccaggtaccagggagttgcatggggcagtgcccgggccgtggcggggggcatgaatttgttgcagggtctgcagtactgagaacagcgtagaaccagtggcgatgggaggaaggggaccggtagagcggggctgggtaagcctccatccagccgggctgagccctggtctccgcagagcctctgcccctcccgagacacccgctacctggtgttagcggtggaccgccctgcgggggcctggcgcggctccgggctggccttgaccctgcagccccgcggagagggtaggtccgcgtggagagggacggggagccgggtcgactgcccccgggcccccagcccctgagccagccgcgtgcccacccaccgcagactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggtgcgcgcaggcaccgggacacggggcaggagcgggcgggggcggcgtggcctcgtggccgctctcaactcctccaattgcgggttccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggaggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggaacgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagc

SEQ ID NO: 4 - protein, 560 aa, Homo sapiens
  ref <308>: DATABASE ACCESSION NUMBER: Genbank/K03474
  ref <309>: DATABASE ENTRY DATE: 1996-05-03
  ref <313>: RELEVANT RESIDUES: (1)..(560)
MRDLPLTSLALVLSALGALLGTEALRAEEPAVGTSGLIFREDLDWPPGIPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR